(19) United States
(12) Patent Application Publication
(10) Pub. No.: US 2023/0357292 A1
(43) Pub. Date: Nov. 9, 2023

(54) MULTI-COORDINATE GOLD-PHOSPHINE COMPOUNDS AND METHOD OF USE

(71) Applicant: University of Kentucky Research Foundation, Lexington, KY (US)

(72) Inventors: Samuel G. Awuah, Lexington, KY (US); Jong Hyun Kim, Lexington, KY (US)

(21) Appl. No.: 18/029,057

(22) PCT Filed: Sep. 29, 2021
(86) PCT No.: PCT/US21/52719

§ 371 Date: Mar. 28, 2023

Related U.S. Application Data
(60) Provisional application No. 63/084,929, filed on Sep. 29, 2020.

(51) Int. Cl. C07F 9/50; C07F 9/58; A61P 35/00; A61K 47/54

(52) U.S. Cl. CPC C07F 9/5045; C07F 9/58; A61P 35/00; A61K 47/545

(57) ABSTRACT

The presently-disclosed subject matter includes a multi-coordinate gold-phosphine compound or a pharmaceutically acceptable salt thereof.

### RELATED APPLICATIONS

This application claims priority from U.S. Provisional Application Ser. No. 63/084,929 filed Sep. 29, 2020, the entire disclosure of which is incorporated herein by this reference.

### GOVERNMENT INTEREST

This invention was made with government support under grant number R01NS112693-01A1 awarded by the National Institutes of Health. The government has certain rights in the invention.

### TECHNICAL FIELD

The presently-disclosed subject matter relates to useful compounds and compositions, and methods of making and methods of using such compounds and compositions. The presently-disclosed subject matter also relates to methods of treating cancer and/or modulating mitochondrial respiration and metabolism using compounds and compositions disclosed herein.

### INTRODUCTION

Platinum drugs, such as cisplatin, remain the first-line treatment option for many cancer types.^{1,2} For example, triple negative breast cancer (TNBC) remains a clinical challenge due to its molecular and genetic heterogeneity, as well as the lack of validated drug targets^{88,89}; however, a treatment approach for TNBC includes platinum agents, with additional treatment options including taxanes, anthracycline, and more recently a combination therapy involving Atezolizumab (PD-L1 inhibitor) and Nab-paclitaxel for metastatic TNBC⁹⁰⁻⁹². Nevertheless, these chemotherapeutic strategies are limited by acquired or intrinsic resistance, lack of potency, toxic side effects, and cross-resistance⁹³. Meanwhile, platinum therapy has been associated with induction of resistance and toxic side effects.³⁻⁶

The drawbacks of platinum therapy has prompted the generation of new metal-based anticancer agents.³⁻⁶ Metal complexes with different mechanisms of action in the biological context hold great promise. The mode of action of metal complexes is usually by covalent modification of biomolecules,⁷⁻⁹ non-covalent interaction with target proteins,¹⁰⁻¹² redox activation by biomolecules,¹³⁻¹⁵ and photosensitizer action.¹⁶ This has propelled the broad application of metal-based compounds in the treatment of diseases such as microbial infection, rheumatoid arthritis, diabetes, and cancer.¹⁷ Alternative metals used in recent metal-based drug discovery include ruthenium,^{18,19} rhenium,^{20,21} osmium,^{22,23} and gold.²⁴⁻²⁷ Additionally, developing metal-based anticancer compounds with a known mechanism of action, well differentiated from cisplatin is essential to overcome cisplatin resistance and improve therapeutic performance.

Accumulating evidence shows that aggressive tumors, including TNBC,⁶¹⁻⁶⁵ have strong dependence on both glycolytic and oxidative pathways. Thus, a promising strategy for developing improved cancer therapy involves targeting mitochondrial metabolism for cancer therapy. For example, it has been shown that combining BACH1 targeting with oxidative phosphorylation electron transport chain (OXPHOS) inhibition by metformin is an effective TNBC therapy⁶⁷. The lack of potency of metformin has prompted several groups to develop superior inhibitors of OXPHOS as therapeutic agents for several diseases including cancer⁶⁸⁻⁷³. A small-molecule, Gboxin, has been demonstrated for use as an inhibitor of OXPHOS to target glioblastoma⁷⁴. This work revealed that aggressive tumors rely on mitochondrial respiration for their energy needs, which can be exploited for therapeutic gains. Together these studies illuminate mitochondria as an attractive target for aggressive cancers.

To target the mitochondria and related mitochondrial processes including, mitochondrial OXPHOS, fission/structure, and mitophagy, rationally designed small-molecules capable of perturbing mitochondria function selectively are required. Gold(I) N-heterocyclic carbene compounds have been demonstrated for use as mitochondria-targeted anticancer agents in cancer cells⁹⁴. There is potential for the gold compounds to depolarize mitochondria membrane potential in cancer cells²⁶. Gold possesses a high redox potential with the ability to react with biological selenols/thiols. Auranofin, an FDA approved drug for rheumatoid arthritis, binds thiols and selenols of proteins including thioredoxin. It has been explored for other medical applications, including clinical trials, as a broad-spectrum antiparasitic or anticancer agent^{95,96}.

Overall, gold compounds are an attractive new class of anticancer agents, which are well tolerated in mammalian systems and possess a differentiated mechanism of action from that of platinum drugs. Efforts to further utilize gold in medical therapy have been limited by the lack of facile gold-based synthetic strategies and corresponding chemically diverse gold-based compound libraries. Thus, while gold compounds are resurging as an important class of therapeutics across a broad range of pathophysiology, major challenges in gold-based bioinorganic chemistry remain, including: (i) the kinetic lability of gold(III) compounds under physiological conditions; (ii) the lack of a rational design of gold-based drugs; (iii) affinity for thiol-rich proteins and enzymes;^{8,28} (iv) incomplete understanding of gold reagent localization in living systems; and (v) difficulty in accessing stable high-valent gold(III) compounds.

Significant progress in understanding the structure and reactivity of selected gold(III) complexes has provided new impetus for the use of gold compounds.^{30-36,59} Previous and ongoing contributions to surmount challenges associated with physiologically relevant gold compounds as anticancer libraries through innovative chemistries³⁷⁻³⁹ continue to expand via the development of new scaffolds including gold-porphyrin,^{9,40-44} gold-phosphine,⁴⁵⁻⁵¹ and cyclometalated gold variations.⁵²⁻⁵⁷ Additional synthetic efforts have led to the generation of [C\hat{ }N]-cyclometalated gold(III) compounds, which present strong sigma-donation of electrons to stabilize the gold center.⁵⁸⁻⁵⁹ Furthermore, the repertoire of gold-based agents was expanded with the synthesis of distorted gold constructs bearing chiral phosphine ligands.^{26,60}

Metastasis is responsible for ˜90% of cancer related deaths. Aggressive tumor types, including TNBC, often lead to metastasis with limited treatment options. This medically unmet need requires innovative drug discovery for improved anticancer therapy. TNBCs adopts increased mitochondrial biogenesis and metabolic activity to promote growth and aggressiveness. Thus, inhibition of mitochondrial activity or uncoupling of mitochondrial respiration is an attractive therapeutic target. However, the clinical efficacy of modulators of mitochondrial dynamics including the electron transport chain and mitochondrial structure is limited by inadequate potency, off-target effects, and poor pharmacokinetics. Potent small-molecules to specifically control mitochondrial function in aggressive tumors holds promise, but none are yet described in the art.

Accordingly, there is a need in the art for compounds, compositions, and methods useful for modulating mitochondrial function and for treating cancer, including aggressive cancers, such as TNBC.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compounds that are gold-based small molecules, which have utility as antitumor agents in vitro and in vivo with distinct modes of action. As disclosed herein, specific inhibition or uncoupling activity of mitochondrial respiration by anticancer gold agents in cancer cells exerts profound antitumor activity in 4T1-tumor bearing animals. Furthermore, compounds as disclosed herein are capable of targeting distinct mitochondrial dynamics, such as mitochondrial structure to exert profound antitumor effects. These compounds are stable toward biological thiols and demonstrate enhanced mitochondrial oxygen consumption ratea and induce proton leaking in aggressive cancer cells. Strikingly, as disclosed herein, in a comparative screening of a reference set of 60 cancer cell lines compiled by the National Cancer Institute (NCI-60) these compounds induce lethality across all tumor types. Whole-cell transcriptomics reveal a broader mechanism of action in addition to the dominant oxidative phosphorylation target supported by quantitative proteomic studies in response to the presently-disclosed compound. The presently-disclosed compound was also tested in a metastatic 4T1 TNBC mouse model and produced significant tumor inhibition. Thus, embodiments of the presently-disclosed subject matter includes compound, compositions, and methods for directly target dysfunctional mitochondria and serve as potent anticancer agents.

The presently-disclosed subject matter includes a compound having the following formula or a pharmaceutically acceptable salt thereof:

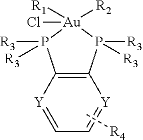

wherein R₁ and R₂, taken together with the gold (Au) to which they are bound, form a polycyclic moiety selected from the group consisting of:

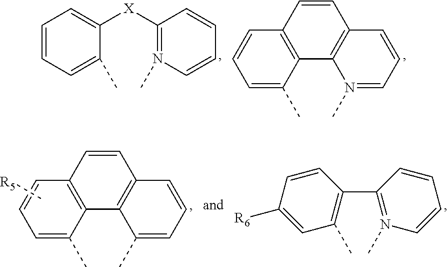

X is CH₂, O, NH, or C═O; Y is C or N; each R₃ is independently selected from the group consisting of H, alkyl, and phenyl; R₄ is H, alkyl, phenyl, or taken together with two of the atoms of the ring to which it is bound, forms a 6-membered ring; R₅ is H, alkyl, or phenyl; and R₆ is H, CH₃, COH, a targeting ligand, or an affinity tag.

In some embodiments of the presently-disclosed subject matter, R₆ is a targeting ligand. In some embodiments of the presently-disclosed subject matter, R₆ is an affinity tag.

In some embodiments of the presently-disclosed subject matter the compound has the following structure:

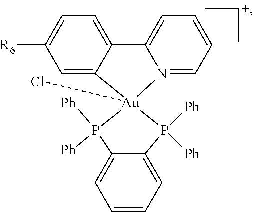

wherein R₆ is selected from the group consisting of CH₃, COH, and H.

In some embodiments of the presently-disclosed subject matter the compound has the following structure:

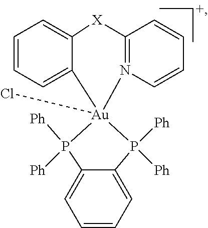

wherein X is selected from the group consisting of CH₂, O, NH, and C═O.

In some embodiments of the presently-disclosed subject matter the compound has the following structure:

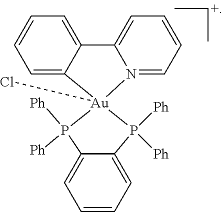

In some embodiments of the presently-disclosed subject matter the compound has the following structure:

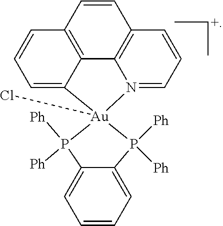

In some embodiments of the presently-disclosed subject matter the compound has the following structure:

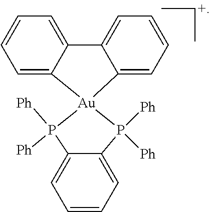

In some embodiments of the presently-disclosed subject matter the compound has the following structure:

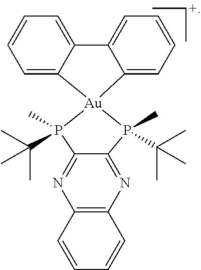

The presently-disclosed subject matter further includes a pharmaceutical composition that includes a compound as disclosed herein and a pharmaceutically-acceptable carrier.

The presently-disclosed subject matter further includes a method of conferring anti-cancer activity to a cancer cell, which involves contacting a cancer cell with an effective amount of a compound or composition as disclosed herein. In some embodiments, the conferring anti-cancer activity results in one or more of inhibiting proliferation of the cancer cell, inhibiting metastasis, and killing the cancer cell. In some embodiments of the method, the cell is a cultured cell. In some embodiments of the method, the cell is in a subject. In some embodiments of the method, the subject is a mammal.

The presently-disclosed subject matter further includes use of a compound or composition as disclosed herein in a medicament for the treatment of a cancer.

The presently-disclosed subject matter further includes a method of modulating mitochondrial function in a cell, comprising: contacting a cell with an effective amount of a compound or composition as disclosed herein. In some embodiments of the method, the cell is a cancer cell. In some embodiments of the method, the cell is a cultured cell. In some embodiments of the method, the cell is in a subject. In some embodiments of the method, the subject is a mammal.

The presently-disclosed subject matter further includes use of a compound or composition as disclosed herein in a medicament for the treatment of a condition involving mitochondrial dysfunction.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1E. Chemical structures of AuPhos compounds. FIG. 1A: Synthetic scheme to access organogold(III) compounds investigated in this study. FIG. 1B-1E: Crystal structures of AuPhos-81, 82, 84, and 83. Outer-sphere solvent molecules are omitted for clarity. Thermal ellipsoids are shown at the 50% probability level. In FIG. 1D, only one representative molecule from the asymmetric unit is shown.

FIG. 2A-2H. Evaluation of cytotoxicity for AuPhos compounds in MDA-MB-231.

FIG. 3A-3H. Evaluation of cytotoxicity for AuPhos compounds H460.

FIG. 4A-4H. Evaluation of cytotoxicity for AuPhos compounds in A2780.

FIG. 5A-5H. Evaluation of cytotoxicity for AuPhos compounds in OVCAR8.

FIG. 6. Evaluation of cytotoxicity for 1,2-Bis(diphenylphosphino)benzene compounds in MDA-MB-231, IC₅₀ is 1.37±0.45 μM. Compared with the AuPhos compounds (Table 1), it shows less cytotoxicity of ˜6 times and confirms the AUPhos compound's superior activity.

FIG. 7A-7C. AuPhos induces apoptosis in breast cancer cells. FIG. 7A. Quadrants displaying apoptotic population of MDA-MB-231 within 15 h of treatment with AuPhos-89 (1 μM). Cells were seeded at a density of 5×10⁵ per well. FIG. 7B. Quadrants displaying apoptotic population of MDA-MB-231 within 3 h of treatment with H₂O₂ (2 mM).

FIG. 7C. Western blotting of apoptotic markers following the exposure of AuPhos-89 (1 μM) at indicated times. Cells were seeded at a density of 5×10⁵ per well. Data is representative of three individual experiments. H₂O₂ was used a positive control.

FIG. 8. HPLC chromatogram of AuPhos-89+GSH (λ=280 nm).

FIG. 9. ¹H NMR spectra (DMSO-d₆) of AuPhos-89 (20 mM), GSH (20 mM), and AuPhos-89 GSH (10 mM).

FIG. 10A-10C. NCI-60 screening result of AuPhos-83 (FIG. 10A), AuPhos-84 (FIG. 10B), and AuPhos-89 (FIG. 10C).

FIG. 11. Summary of the NCI-60 human tumor cell line screen. AuPhos 83, 84, and 89 show superior activity across the panel of cell lines tested including breast. Numbers in parentheses represent the number of cell lines tested for each indication. The breast panel includes TNBC (MDA-MB-231, BT549, Hs578T, MDA-468) and luminal cell lines (MCF7, T47D).

FIG. 12A-12B. Whole-cell transcriptomics. FIG. 12A: Representative heat map of DEGs in response to AuPhos-89. FIG. 12B: KEGG analysis plots outlining varying pathways perturbed upon treatment with AuPhos-89. MDA-MB-231 cells were treated with AuPhos-89 (1 μM for 12 h) and pure RNA isolated for sequencing. Data are representative of two independent replicates.

FIG. 13. Whole cell (OVCAR8) uptake results from auranofin (5 μM) and complexes AuPhos-83, 84, and 89 (10 μM). Cells were incubated with compounds for 15 h.

FIG. 14A-14C. Cellular and mitochondrial uptake study. FIG. 14A: Whole-cell uptake results of representative AuPhos compounds in MDA-MB-231 cells. FIG. 14B: Mitochondrial accumulation of AuPhos-89 in breast cancer epithelial cells. FIG. 14C: Mitochondrial accumulation of AuPhos-89 in normal colon epithelial cells (NCM 460). Data are a mean of three independent replicates. For (FIG. 14B), unpaired t-test, *P<0.0001, for (FIG. 14C), ordinary one-way ANOVA, **P<0.01, ***P<0.001, ****P<0.0001

FIG. 15A-15G Effect of AuPhos on bioenergetics. FIG. 15A: OCR mediated by complex I (State IV) from the effect of AuPhos-89 on isolated liver mitochondria in the presence of oligomycin. FIG. 15B: OCR mediated by complex I (State IV) from the effect of AuPhos-89 on isolated liver mitochondria in the presence of oligomycin and Ca²⁺. FIG. 15C: Schematic diagram of the mitochondrial respiration experiment. FIG. 15D: Proton leak extrapolated from the mito-stress test of MDA-MB-231 cells treated with AuPhos-89, FIG. 15E: ATP production extrapolated from the mito-stress test of AuPhos-89 treated MDA-MB-231 cells. FIG. 15F: Changes in the membrane potential of MDA-MB-231 cells depending on the concentration of AuPhos-89 using TMRE assay and FCCP as control. FIG. 15G: Mitochondrial membrane potential measured by FACS of MDA-MB-231 cells, control (left), AuPhos-89-treated (middle) using JC-1 assay. Ordinary one-way or two-way ANOVA, *P<0.01 and ****P<0.0001.

FIG. 16A-16C. Cellular responses to AuPhos-89. FIG. 16A: Cell cycle distribution by PI staining: br graph representing the percentage of cells in G1, G2, and S. MDA-MB-231 cell bars represent an average of 3 measurements. Error bars represent means SD. FIG. 16B: Time-dependent activation of AMPKα and of MDA-MB-231 treated with AuPhos-89 (1 μM) and analyzed by immunoblotting. FIG. 16C: TMT-labelled quantitative proteomics. Differentially expressed protein data; only Log₂ FC values more than 1 and less than −1 were extracted and displayed. Proteins with high values are shown in the table. MDA-MB-231 cells were treated with AuPhos-89 (1 μM, 12 h).

FIG. 17A-17F. Antitumor effect induced by AuPhos-89. FIG. 17A: Impact of AuPhos-89 on the tumor volume of 4T1 (1 million cells inoculated, n=5). Unpaired t-test, *P<0.05. FIG. 17B: Weight of mice (n=3) following intravenous administration of AuPhos-89 and observed over 19 days. FIG. 17C: Tissue biodistribution of AuPhos-89 in mice as determined by GF-AAS, which measures gold content. The compound was administered by intravenous injection and at indicated time points, tissues were collected after mice (n=3) were euthanized. FIG. 17D: Hematoxylin and eosin (H&E) staining indicates reduced cellularity and proliferation in tumors treated with AuPhos-89. Liver metastasis is observed in control mice with no palpable metastatic lesions in treated mice.

FIG. 17E: Comparative in vivo efficacy study of AuPhos-89 and cisplatin. Impact of AuPhos-89 and cisplatin on the tumor volume of 4T1 (two million cells inoculated, n=5). Ordinary one-way ANOVA test, *P<0.05. FIG. 17F: Weight of mice following intraperitoneal administration of AuPhos-89 and cisplatin.

FIG. 18 shows UV-vis absorption spectra of AuPhos with 10 mM L-Glutathione.

FIG. 19A-19B. Compounds belonging to the AuPhos class demonstrate IC₅₀s of 150-400 nM in MDA-MB-231 cells. Detailed mitochondrial studies using mouse liver mitochondria and MDA-MB-231 cells support an uncoupling OXPHOS activity by AuPhos-89 in a dose-dependent manner.

FIG. 20A-20B. Two million 4T1 cells were injected subcutaneously into mouse flanks treated three times a week by intraperitoneal injection of vehicle control, cisplatin, or Au-Phos-89, beginning three days after inoculation (n=5). FIG. 20C-E. Images of excised tissue and organs after 12 days.

FIG. 21A-21F. Stability in PBS over 24 hours of AuPhos-84 (25 μM) (FIG. 21A), AuPhos-85 (25 μM)(FIG. 21B), and AuPhos-89 (25 μM)(FIG. 21C); and stability in RPMI 1640 over 24 h, for AuPhos-84 (25 μM)(FIG. 21D), AuPhos-85 (25 μM)(FIG. 21E), and AuPhos-89 (25 μM)(FIG. 21F).

FIG. 228A-22D. Chemical structures of additional AuPhos compounds. FIG. 22A: Chemical structures of various AuPhos compounds (Formulae I-IV). FIG. 22B: Chemical structures exemplary Compound A and Compound B. FIG. 22C-22D: Crystal structures of Compound A and Compound B. Outer-sphere solvent molecules are omitted for clarity. Thermal ellipsoids are shown at the 50% probability level.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compounds, compositions, and methods useful for modulating mitochondrial function in a cell and for treating cancer.

The presently-disclosed subject matter includes a compound having the following formula or a pharmaceutically acceptable salt thereof:

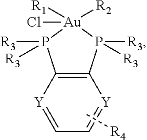

wherein R₁ and R₂, taken together with the gold (Au) to which they are bound, form a polycyclic moiety selected from the group consisting of:

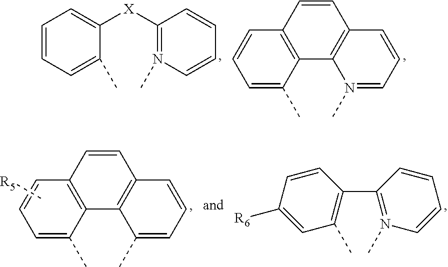

X is CH₂, O, NH, or C═O; Y is C or N; each R₃ is independently selected from the group consisting of H, alkyl, and aryl; R₄ is H, alkyl, aryl, or taken together with two of the atoms of the ring to which it is bound, forms a 6-membered ring; R₅ is H, alkyl, or aryl; and R₆ is H, CH₃, COH, a targeting ligand, or an affinity tag.

In some embodiments of the presently-disclosed subject matter, R₆ is a targeting ligand. Targeting ligands for directing compounds to a cancer cell are well known to those of ordinary skill in the art, with examples including, but not limited to folic acid, mannose, an antibody (e.g., anti-CD22, anti-CD25, anti-EpCAM, etc.), an aptamer (e.g., SYL3C for targeting EpCAM, AptA for targeting Mucin1, etc.), or a polypeptide (e.g., arginine-glycine-aspartic acid (RGD) motif, yclic 9-mer iRGD (CRGDKGPDC), other polypeptides targeting integrins, epidermal growth factor receptor (EGFR), somatostatin receptors (SSTRs), gonadotropin-releasing hormone receptor (GnRH-R), bombesin (Bn) receptors, G protein-coupled receptors (GPCRs), etc.).

In some embodiments of the presently-disclosed subject matter, R₆ is an affinity tag. Affinity tags are well known to those of ordinary skill with examples including, but not limited to, polyhistidine tag, glutathione-S-transferase (GST) tag, Strep-tag (streptavidin-binding tag), almodulin-binding peptide (CBP) tag, chitin-binding domain (CBD), FLAG tag, HA tag, c-Myc tag, etc.

In some embodiments of the presently-disclosed subject matter the compound has the following structure:

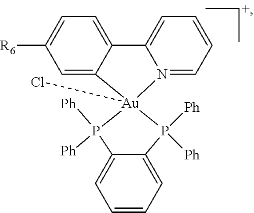

wherein R₆ is selected from the group consisting of CH₃, COH, and H.

In some embodiments of the presently-disclosed subject matter the compound has the following structure:

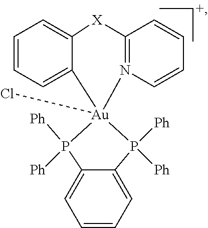

wherein X is selected from the group consisting of CH₂, O, NH, and C═O.

In some embodiments of the presently-disclosed subject matter the compound has the following structure:

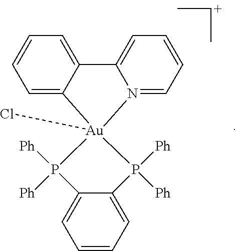

In some embodiments of the presently-disclosed subject matter the compound has the following structure:

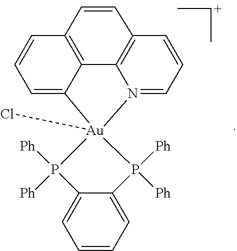

In some embodiments of the presently-disclosed subject matter the compound has the following structure:

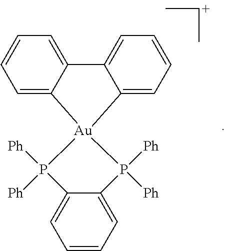

In some embodiments of the presently-disclosed subject matter the compound has the following structure:

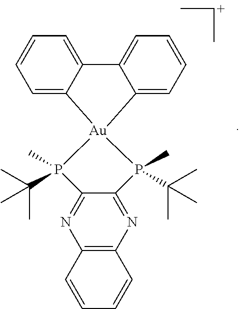

The presently-disclosed subject matter further includes a pharmaceutical composition that includes a compound as disclosed herein and a pharmaceutically-acceptable carrier.

The presently-disclosed subject matter further includes a method of conferring anti-cancer activity to a cancer cell, which involves contacting a cancer cell with an effective amount of a compound or composition as disclosed herein. The cancer cell can be any type of cancer cell, with examples including, but not limited to leukemia, non-small cell lung cancer, colon cancer, central nervous system (CNS) cancer, melanoma, ovarian cancer, renal cancer, prostate cancer, or breast cancer.

In some embodiments, the conferring anti-cancer activity results in one or more of inhibiting proliferation of the cancer cell, inhibiting metastasis, and killing the cancer cell.

In some embodiments of the method, the cell is a cultured cell. In some embodiments of the method, the cell is in a subject. In some embodiments of the method, the subject is a mammal.

The presently-disclosed subject matter further includes use of a compound or composition as disclosed herein in a medicament for the treatment of a cancer.

The presently-disclosed subject matter further includes a method of modulating mitochondrial function in a cell, comprising: contacting a cell with an effective amount of a compound or composition as disclosed herein. In some embodiments of the method, the cell is a cancer cell. In some embodiments of the method, the cell is a cultured cell. In some embodiments of the method, the cell is in a subject. In some embodiments of the method, the subject is a mammal.

The presently-disclosed subject matter further includes use of a compound or composition as disclosed herein in a medicament for the treatment of a condition involving mitochondrial dysfunction.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments ±5%, in some embodiments±1%, in some embodiments±0.5%, in some embodiments±0.1%, in some embodiments±0.01%, and in some embodiments±0.001% from the specified amount, as such variations are appropriate to perform the disclosed method.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

In some aspects of the disclosed methods, the subject has been diagnosed with a need for treatment of a disease state associated with cancer. In some aspects of the disclosed method, the subject has been diagnosed with a need for inhibition of metastasis.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “subject” refers to a target of administration or medical procedure. The subject of the herein disclosed methods can be a human or animal. The subject may also be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder that creates intestinal mucosal injury” means having been subjected to a physical examination by a person of skill, for example, a physician, and found to have a condition that can be diagnosed or treated by a compound or composition that can promote healing of intestinal mucosal injury. Such a diagnosis can be in reference to a disorder, such as IBD or COVID induced enteritis, and the like, as discussed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by a formula —C(O)H. Throughout this specification “C(O)” is a short hand notation for a carbonyl group, i.e., C═O.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by a formula —C(O)OH.

The term “ester” as used herein is represented by a formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by a formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by a formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by a formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by a formula —OH.

The term “ketone” as used herein is represented by a formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by a formula —N₃.

The term “nitro” as used herein is represented by a formula —NO₂.

The term “nitrile” as used herein is represented by a formula —CN.

The term “silyl” as used herein is represented by a formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by a formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by a formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by a formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by a formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by a formula —SH.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compounds disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

In some aspects, a structure of a compound can be represented by a formula:

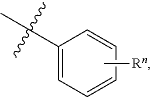

which is understood to be equivalent to a formula:

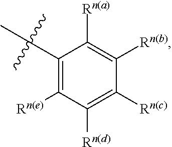

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{(b)} is not necessarily halogen in that instance. Likewise, when a group R is defined as four substituents, R is understood to represent four independent substituents, R^{a}, R^{b}, R^{c}, and R^{d}. Unless indicated to the contrary, the substituents are not limited to any particular order or arrangement.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Materials and Instrumentation

All chemicals were purchased from Sigma-Aldrich and used without further purification. Tetrachloroauric acid (HAuCl₄·3H₂O) was purchased from NANOPARTZ and stored in a glovebox or dessicator before use as received. ACS grade solvents were purchased from Pharmco-Aaper and used without further purification or drying. Deuterated solvents were purchased from Cambridge Isotope Laboratories and used as received. Silica gel for column chromatography (Silicycle, P/N: R10030B SiliaFlash® F60, Size: 40-63 μm, Canada) was purchased from Silicycle. Aluminum backed silica-gel plates (20×20 cm²) were purchased from Silicycle (TLA-R10011B-323) and utilized for analytical thin-layer chromatography (TLC).

All reactions were insensitive to air or moisture, as a result, they were carried out under standard atmospheric conditions without air-sensitive techniques or drying agents. Reactions were carried out in round-bottom flasks or scintillation vials equipped with Teflon-coated magnetic stir bars for stirring non-homogenous reaction mixtures. Reactions were monitored by NMR and TLC, and the TLC plates visualized under low-wavelength light (254 nm) or stained with iodine on Silica. All compound purification was performed using silica-gel chromatography, employing CombiFlash® Rf+ Lumen, Teledyne ISCO. Filtrations were carried out using medium-porosity ceramic funnels. Removal of solvents in vacuo was performed using a Büchi rotary evaporator and further drying was achieved via a Schlenk line at ˜120 mTorr using a dynamic vacuum pump.

¹H, ¹³C (¹H-decoupled), and ³¹P (¹H-decoupled) NMR spectra were recorded on a Varian Unity 400 MHz NMR spectrometer with a Spectro Spin superconducting magnet at the University of Kentucky NMR facility in the Department of Chemistry. Chemical shifts in ¹H and ¹³C NMR spectra were internally referenced to solvent signals (¹H NMR: CDCl₃ at S=7.26 ppm; 13C NMR: CDCl₃ at S=77.16 ppm), and those in ³¹P NMR spectra, which were run in CDCl₃, were externally referenced to 85%_{H3PO4} in D₂O at S=0 ppm.

High-resolution mass spectra (HRMS) were obtained using a Waters Synapt G2 HD mass spectrometer. Samples were directly injected into the instrument at 50 μL/min and ionized with ElectroSpray Ionization (ESI) in the positive mode. The source parameters were: capillary=2.8 kV, sampling cone=40, extraction cone: 5.0, source temperature=80° C., desolvation temperature=150° C., and desolvation gas flow=500 L/h. Mass spectrometry experiments and analysis were conducted at the Central Analytical Laboratory at the University of Colorado, Boulder.

In addition to spectroscopic characterization, bulk purity of all new compounds was assessed by combustion elemental analysis for C, H, N. Elemental analysis was carried out at the Atlantic Microlab (Norcross, GA) using Perkin Elmer 2400 Series II Autoanalyzers and Carlo Erba Model 1108 Analyzers. Instrument specifications list a precision of ±0.3 percent.

### Example 2: X-Ray Crystallography

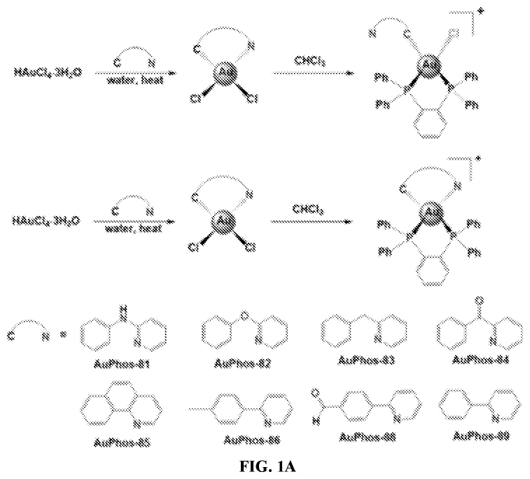

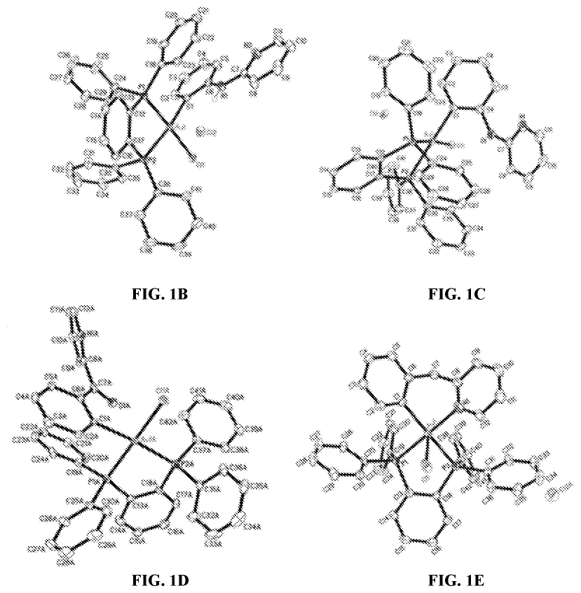

Crystal for AuPhos-82. 83, 84 and 85 were grown at 4° C. or room temperature from a vapor diffusion of either DMF into diethylether or acetone into THF solution of AuPhos compounds. Suitable crystals were selected by microscopic examination through crossed polarizers, mounted on a fine glass fiber in polyisobutane oil, and cooled to 90 K under a stream of nitrogen. A Bruker-AXS D8 Venture dual microsource diffractometer was used to collect the diffraction data using MoKα radiation (λ=0.71073 Å) from the crystal. The raw data were integrated, scaled, merged, and corrected for Lorentz-polarization effects using the APEX3 package⁸⁰. Absorption correction was performed by SADABS⁸¹⁻⁸² within APEX3. Space group determination and structure solution were carried out with SHELXT, and refinement used SHELXL-2017⁸³⁻⁸⁴. All non-hydrogen atoms were refined with anisotropic displacement parameters. Hydrogen atoms were placed at calculated positions and refined using a riding model with their isotropic displacement parameters (Uᵢₛₒ) derived from the atom to which they were attached. The structures, deposited in the Cambridge Structural Database, were checked with an R-tensor⁸⁵, by PLATON⁸⁶, and were further validated using CheckCIF⁷. See FIGS. 1A-1E and Table 1-5 for structure details.

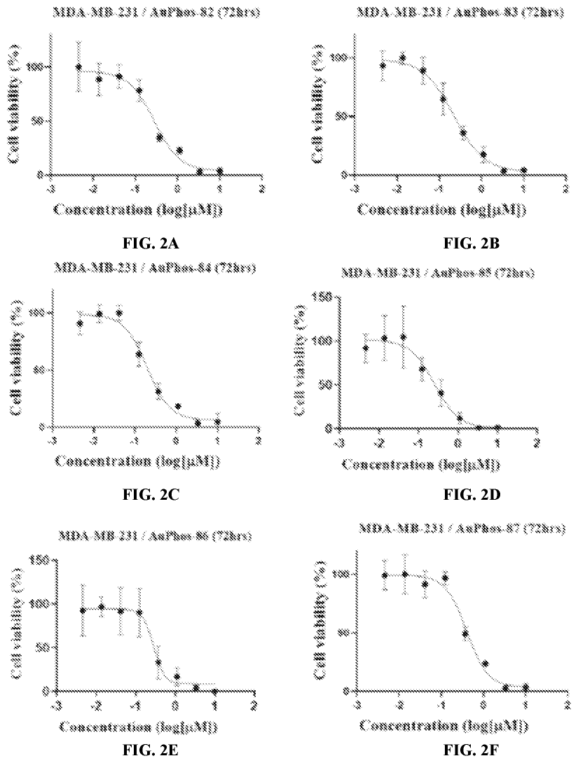

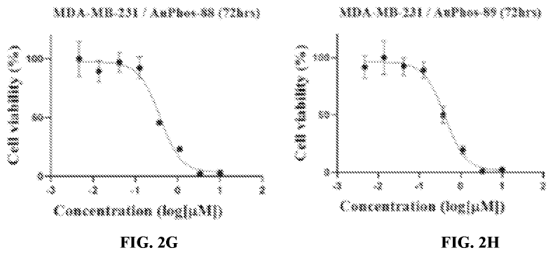

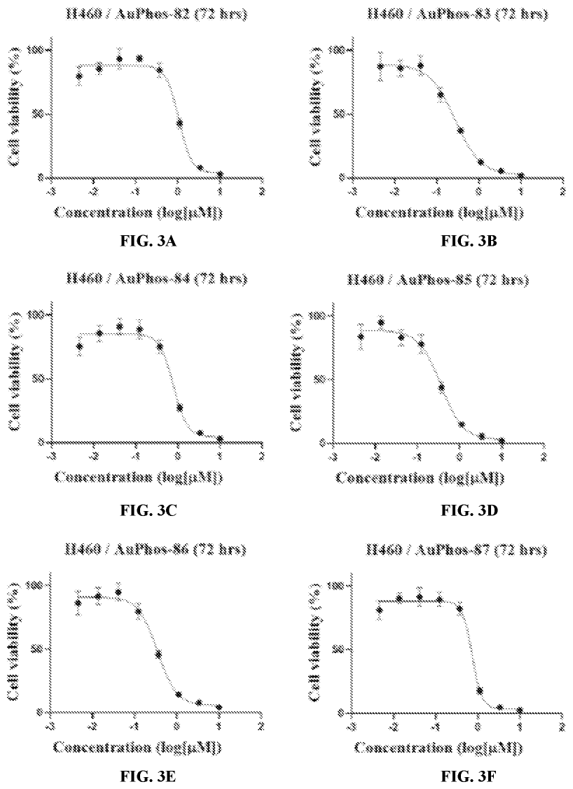

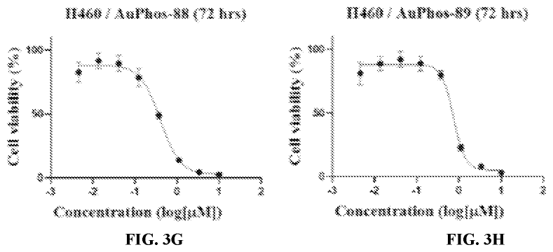

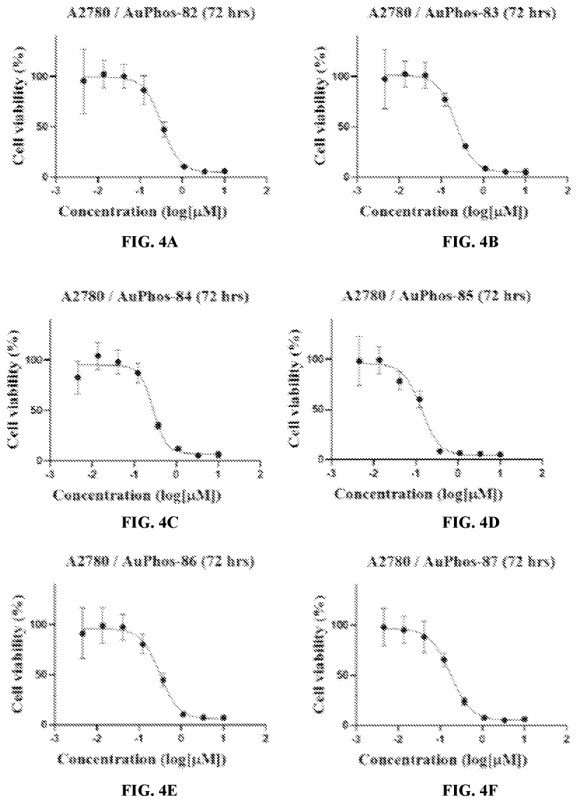

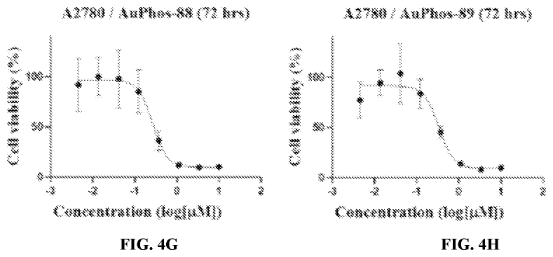

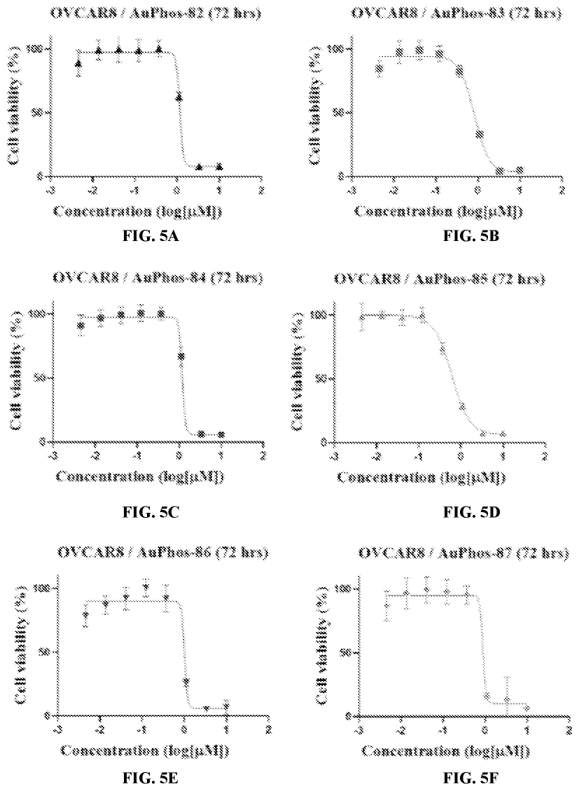

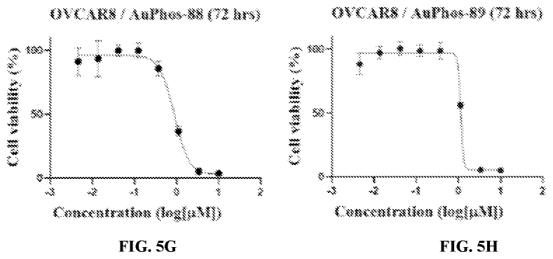

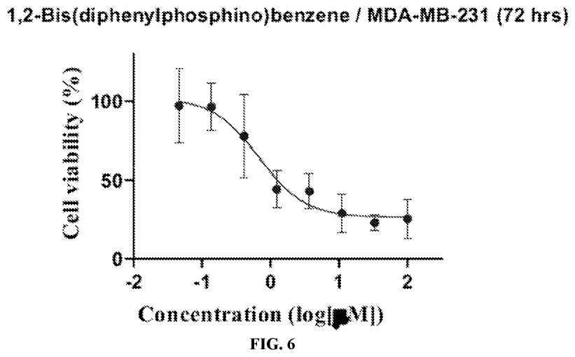

TABLE 1
_{IC50}values for AuPhos compounds across a panel of cell lines. Cells
were seeded at a density of 4,000 cells/well and treated for 72 h. _{IC50}values
are plotted as the mean ± SD (n = 3). Full dose response
curves can be found in the supporting information (FIG. 2-6).

| | IC₅₀ (μM) | | | |
|---|---|---|---|---|
| | MDA-MB-231 | H460 | A2780 | OVCAR8 |
| | (breast) | (lung) | (ovarian) | (ovarian) |
| AuPhos-82 | 0.22 ± 0.20 | 1.04 ± 0.17 | 0.34 ± 0.04 | 0.85 ± 0.06 |
| AuPhos-83 | 0.22 ± 0.07 | 0.27 ± 0.06 | 0.22 ± 0.01 | 0.89 ± 0.14 |
| AuPhos-84 | 0.24 ± 0.16 | 0.76 ± 0.15 | 0.25 ± 0.06 | 1.24 ± 0.14 |
| AuPhos-85 | 0.26 ± 0.06 | 0.34 ± 0.07 | 0.15 ± 0.03 | 0.64 ± 0.11 |
| AuPhos-86 | 0.29 ± 0.04 | 0.34 ± 0.07 | 0.33 ± 0.04 | 0.82 ± 0.44 |
| AuPhos-87 | 0.45 ± 0.19 | 0.71 ± 0.09 | 0.18 ± 0.04 | 0.82 ± 0.76 |
| AuPhos-88 | 0.37 ± 0.21 | 0.40 ± 0.03 | 0.27 ± 0.02 | 0.85 ± 0.07 |
| AuPhos-89 | 0.45 ± 0.14 | 0.72 ± 0.16 | 0.34 ± 0.07 | 1.16 ± 0.04 |

TABLE 2
Crystal data and structure refinement for compound AuPhos-81.

| | AuPhos-81 |
|---|---|
| Empirical formula | C₄₁H₃₃AuCl₂N₂P₂ |
| Formula weight | 883.5 |
| Temperature | 90.0(2) K |
| Crystal system, space group | Monoclinic, P21/c |
| Unit cell dimensions | a = 10.4667(2) Å alpha = 90° |
| | b = 16.4519(4) Å beta = 96.182(1)° |
| | c = 23.6049(6) Å gamma = 90° |
| Volume | 4041.06(16) Å 3 |
| Z, Calculated density | 4, 1.452 Mg/m³ |
| Absorption coefficient | 3.881 mm⁻¹ |
| F(000) | 1744 |
| Crystal size | 0.160 × 0.120 × 0.110 mm |
| Theta range for data collection | 2.054 to 27.520° |
| Limiting indices | −13 ≤ h ≤ 12, −21 ≤ k ≤ |
| | 21, −30 ≤ 1 ≤ 30 |
| Reflections collected/unique | 68155/9298 [R(int) = 0.0394] |
| Completeness to theta = 25.242 | 100.00% |
| Absorption correction | Semi-empirical from equivalents |
| Max. and min. transmission | 0.000 and 0.000 |
| Refinement method | Full-matrix least-squares on F2 |
| Data/restraints/parameters | 9298/0/435 |
| Goodness-of-fit on F2 | 1.087 |
| Final R indices [I > 2σ(I)] | R1 = 0.0180, wR2 = 0.0402 |
| R indices (all data) | R1 = 0.0207, wR2 = 0.0409 |
| Extinction coefficient | 0.00053(4) |
| Largest diff. peak and hole | 0.572 and −0.724 e.Å⁻³ |

TABLE 3
Crystal data and structure refinement for compound AuPhos-82.

| | AuPhos-82 |
|---|---|
| Empirical formula | C₄₄H₃₉AuCl₂N₂O₂P₂ |
| Formula weight | 957.58 |
| Temperature | 90.0(2) K |
| Wavelength | 0.71073 Å |
| Crystal system, space group | Monoclinic, P21/n |
| Unit cell dimensions | a = 10.4749(2) Å alpha = 90° |
| | b = 16.5319(4) Å beta = 93.007(1)° |
| | c = 23.0359(7) Å gamma = 90° |
| Volume | 3983.63(17) Å 3 |
| Z, Calculated density | 4, 1.597 Mg/m³ |
| Absorption coefficient | 3.947 mm⁻¹ |
| F(000) | 1904 |
| Crystal size | 0.150 × 0.120 × 0.020 mm |
| Theta range for data collection | 2.096 to 27.502° |
| Limiting indices | −13 ≤ h ≤ 12, −21 ≤ k ≤ |
| | 21, −29 ≤ 1 ≤ 29 |
| Reflections collected/unique | 57806/9157 [R(int) = 0.0560] |
| Completeness to theta = 25.242 | 100.00% |
| Absorption correction | Semi-empirical from equivalents |
| Max. and min. transmission | 0.746 and 0.632 |
| Refinement method | Full-matrix least-squares on F2 |
| Data/restraints/parameters | 9157/56/523 |
| Goodness-of-fit on F2 | 1.035 |
| Final R indices [I > 2σ(1)] | R1 = 0.0249, wR2 = 0.0419 |
| R indices (all data) | R1 = 0.0371, wR2 = 0.0451 |
| Extinction coefficient | 0.00030(3) |
| Largest diff. peak and hole | 0.635 and −0.649 e.Å⁻³ |

TABLE 4
Crystal data and structure refinement for compound AuPhos-83.

| | AuPhos-83 |
|---|---|
| Empirical formula | C₄₈H₄₆AuCl₂NO₂P₂ |
| Formula weight | 998.66 |
| Temperature | 90.0(2) K |
| Wavelength | 0.71073 Å |
| Crystal system, space group | Monoclinic, P21/c |
| Unit cell dimensions | a = 8.9901(2) Å alpha = 90 deg. |
| | b = 17.6443(4) Å beta = 95.938(1) deg. |
| | c = 27.1668(7) Å gamma = 90° |
| Volume | 4286.19(18) Å³ |
| Z, Calculated density | 4, 1.548 Mg/m³ |
| Absorption coefficient | 3.671 mm⁻¹ |
| F(000) | 2000 |
| Crystal size | 0.130 × 0.110 × 0.100 mm |
| Theta range for data collection | 2.309 to 27.522° |
| Limiting indices | −11 ≤ h ≤ 11, −22 ≤ k ≤ |
| | 22, −35 ≤ 1 ≤ 35 |
| Reflections collected/unique | 67263/9841 [R(int) = 0.0605] |
| Completeness to theta = 25.242 | 99.90% |
| Absorption correction | Semi-empirical from equivalents |
| Max. and min. transmission | 0.694 and 0.621 |
| Refinement method | Full-matrix least-squares on F² |
| Data/restraints/parameters | 9841/85/526 |
| Goodness-of-fit on F² | 1.067 |
| Final R indices [I > 2σ(I)] | R1 = 0.0296, wR2 = 0.0534 |
| R indices (all data) | R1 = 0.0463, wR2 = 0.0566 |
| Extinction coefficient | n/a |
| Largest diff. peak and hole | 0.675 and −0.814 e.Å³ |

TABLE 5
Crystal data and structure refinement for compound AuPhos-84.

| | AuPhos-84 |
|---|---|
| Empirical formula | C₄₂H₃₂AuCl₂NOP₂ |
| Formula weight | 896.49 |
| Temperature | 90.0(2) K |
| Wavelength | 0.71073 Å |
| Crystal system, space group | Triclinic, P1 |
| Unit cell dimensions | a = 12.0282(6) Å alpha = 97.253(2)° |
| | b = 16.7453(8) Å beta = 95.135(2)° |
| | c = 20.3891(11) Å gamma = 90.405° |
| Volume | 4056.8(4) Å³ |
| Z, Calculated density | 4, 1.468 Mg/m³ |
| Absorption coefficient | 3.868 mm⁻¹ |
| F(000) | 1768 |
| Crystal size | 0.140 × 0.090 × 0.070 mm |
| Theta range for data collection | 2.206 to 27.530° |
| Limiting indices | −15 ≤ h ≤ 15, −21 ≤ k ≤ |
| | 21, −26 ≤ 1 ≤ 26 |
| Reflections collected/unique | 44289/44289 [R(int) = ?] |
| Completeness to theta = 25.242 | 96.00% |
| Absorption correction | Semi-empirical from equivalents |
| Max. and min. transmission | 0.746 and 0.650 |
| Refinement method | Full-matrix least-squares on F2 |
| Data/restraints/parameters | 9841/85/526 |
| Goodness-of-fit on F2 | 1.058 |
| Final R indices [I > 2σ(I)] | R1 = 0.0529, wR2 = 0.1550 |
| R indices (all data) | R1 = 0.0727, wR2 = 0.1696 |
| Extinction coefficient | n/a |
| Largest diff. peak and hole | 1.939 and −2.846 e.Å⁻³ |

### Example 3: Stability Experiment of AuPhos-89 with L-GSH (HPLC Chromatogram)

CH3CN (ACS reagent grade) solvent was degassed overnight using a sonicator and then filtered with a 0.22 μm pore size filter paper before use. All spectra were recorded using an Agilent Technologies 1100 series HPLC instrument and an Agilent Phase Eclipse Plus C18 column (4.6 mm×100 mm; 3.5 μm particle size). AuPhos-89 and glutathione were prepared as 1 mM stock in DMSO and water, respectively. Equimolar amounts were mixed and the solutions were measured at 0, 1, 3, and 24 h by HPLC. The parameters used in the HPLC were as follows: flow rate, 1 mL/min; λ=280 nm; eluent A=H2O with 0.1% TFA; Eluent B═CH3CN with 0.05% HCOOH; Solvent gradient: 0 min (100:0 H₂O:CH3CN), 10 min (0:100 H₂O:CH3CN), 15 min (100:0 H₂O:CH3CN).

### Example 4: Reactivity with L-GSH (¹H NMR Spectroscopy)

Stock solutions comprised of a 1 mL, 20 mM solution of AuPhos-89 in DMSO-d6. The solution of L-GSH (20 mM) was sonicated for 5 minutes to dissolve the reagent in DMSO-d6. 1 mL of each solution were mixed in a 1:1 ratio to produce a final concentration of 10 mM in DMSO-d6. The solution was then analyzed by 1H NMR spectroscopy. The solution was further analyzed at different time intervals, t=1 h, 6 h, 12 h, and 24 h.

### Example 5: Cell Lines and Cell Culture Conditions

All cell lines (OVCAR8, A2780, H460, and MDA-MB-231) were cultivated in RPMI or DMEM supplemented with 10% FBS, 1% penicillin/streptomycin, and 1% amphotericin. All cells were grown at 310K in a humidified atmosphere containing 5% CO2.

### Example 6: Cell Viability Assay

Various established human ovarian, lung, and breast cancer cell lines were seeded in 96-well plates (4000 cells/well) and were incubated with RPMI 1640 or DMEM (100 μL) supplemented with 10% FBS, 1% penicillin/streptomycin, and 1% amphotericin for 24 h and at 37° C. They were then treated with AuPhos compounds at increasing concentrations for 72 h from stock solutions following serial dilutions, in which AuPhos stock solutions were prepared in DMSO/media mixture (% v/v=20:80). Thereafter, cellular viability was assessed via the established crystal violet colorimetric assay. In brief, cells were fixed with 50 μL of a 1% glutaraldehyde solution and placed in an incubator (37° C., 5% CO2) for 1 hour. The plates were washed with water by running them under a gentle flow of tap water. The plates were air-dried, and crystal violet reagent was added to each well and allowed to incubate at room temperature for 20 min. The plates were washed with water and dried again. Methanol (200 μL/well) was added to each well, and the measurements of absorbance were subsequently performed using a Biotek Synergy H1 Plate Reader at 570 nm (peak absorbance).

### Example 7: Whole-Cell Uptake Studies

OVCAR8 Cells and MDA-MB-231 (5×10⁵) were seeded in a 6-well plate and incubated overnight for attachment. Cells were then incubated with the test compounds (1 μM or 5 μM) in fresh media (RPMI or DMEM) and subsequently incubated for a given time (15 h for OVCAR8 and 18 h for MDA-MB-231, respectively) at 37° C. The media were then removed, and cells were collected via trypsinization. Cells were then washed with PBS (3×1 mL). The cells were digested by adding 0.1-0.2 mL of 70% HNO₃, and briefly sonicated. The cell solution was then diluted to an appropriate concentration using DI water. The gold content was analyzed by GF-AAS to obtain the whole cell uptake after quantification.

### Example 8: Mitochondrial Uptake Assay

To measure the mitochondrial uptake, MDA-MB-231 and NCM 460 cells (20×10⁶) were treated with 1 or 5 μM of AuPhos-89 at 37° C. for 12 h or 18 h. The medium was removed, and the cells were washed with PBS solution (1 mL×3), harvested, and centrifuged. The mitochondria extraction kit (ThermoFisher Scientific) was used to extract the mitochondria of the cells according to the manufacturer's protocol. The separated mitochondria pellets were mineralized with 70% HNO₃ and then diluted with DI water as needed. The gold content was analyzed by GF-AAS. Cellular gold levels were expressed as pmol of Au per million cells.

### Example 9: Differential Gene Expression Using RNA-Sequencing

MDA-MB-231 cells were seeded in petri-dishes (100 mm×15 mm) and allowed to grow to 85% confluency. The cells were then treated with AuPhos-89 at a concentration of 1 i.IM for 12 h at 37° C. Cells were harvested and 1×10⁷ cells were collected. High quality RNA was isolated using RNA Qiagen kit following manufacturer's protocol and subsequently sent to Novogene® for RNA-sequencing and analysis. Prior to analysis samples were required to pass three tests before library construction: 1) nanodrop for RNA purity_{(OD260/OD280)}, 2) agarose gel electrophoresis for RNA integrity and potential contamination, and 3) Agilent 2100 check RNA integrity. Next, the NEB library was constructed from mRNA enrichment and fragmentation, followed by reverse transcription, second strand cDNA synthesis, end repair, addition of adaptor, and finally amplification with PCR. After library construction, qPCR was used to accurately quantify the library effective concentration (>2 nM), in order to ensure the library quality. Raw reads were removed via the following parameters: 1) remove reads containing adaptors, 2) remove reads containing N>10% (N represents bases that could not be determined), 3) the Qscore (Quality value) of over 50% bases of the read was £5. Novogene® then uses STAR to accomplish the mapping reads to the reference genome. Gene expression level is then estimated by the abundance of transcripts (count of sequencing) that mapped to genome or exon where read counts are proportional to gene expression level, gene length and sequencing depth. Samples are then subjected to analysis using Pearson's correlation coefficient and principal component analysis for statistical significance.

### Example 10: Mitochondrial Membrane Potential (JC-1)

MDA-MB-231 cells were plated at a density of 5×10⁵ cells/plate in a 6 well plate and allowed to adhere overnight at 37° C. AuPhos-89 was prepared as a stock in DMSO/DMEM (% v/v=20:80) and added at a final concentration of 10 μM. The cells were incubated (37° C., 5% CO2) for 1 h at this concentration. Carbonyl cyanide 3-chlorophenylhydrazone (CCCP) was prepared as a stock in DMSO and added at a final concentration of 100 μM, and the cells were treated for 1 h. It was used as a positive control. After the indicated treatment time, a working solution of the JC-1 dye (Cayman Chemicals, Cat. #15003) was prepared by adding 100 μL of dye into 900 μL of DMEM. Note: the working solution of JC-1 should always be prepared fresh and not stored for long-term use. Then, 100 μL/mL of working solution of the JC-1 dye was added to the cells and incubated at 37° C. for 20 minutes. The cells were harvested and resuspended in 2 mL PBS. The cells were analyzed on a flow cytometer with 488 nm excitation and appropriate emission filters. Gated cells, excluded debris and the CCCP-treated sample was used to performed standard compensation.

### Example 11: Mitochondrial Metabolism Analysis with Seahorse XF96

Analysis. The initial step of Seahorse XF96 analysis included optimization of the cell density. In this stage, isolated mitochondria (30 μg) or MDA-MB-231 cells were seeded at a range of densities from 2000 cells/well to 100,000 cells/well, followed by optimization of the FCCP injection concentration used (0.6 μM of 1.2 μM). The optimum conditions were determined to be 30,000 cells/well and an FCCP injection concentration of 0.6 μM. All Seahorse XF96 experiments with MDA-MB-231 were performed under these conditions. The cells were seeded the night prior to the experiment with a final volume of 100 μL and incubated overnight at 37° C. AuPhos-89 was prepared as a stock in DMSO/DMEM (% v/v=20:80) and diluted to a working concentration of 100 μM with Seahorse XF96 assay buffer and then subsequently serial diluted by 10× to achieve multiple concentrations. The assay was performed using a pneumatic injection method of AuPhos-89, with the final injection concentrations of 0.01, 0.1, 1, and 10 μM. This was followed by injection of oligomycin (1.5 μM), FCCP (0.6 μM) and rotenone/antimycin A (0.5 μM).

### Example 12: Cell Cycle Analysis

MDA-MB-231 cells were seeded at a density of 5×10⁵ cells/well in a 6-well clear bottom plate with a final media volume of 2 mL and allowed to adhere overnight 37° C. AuPhos-89 was prepared fresh as a stock in DMSO/DMEM (% v/v=20:80) and added at a final concentration of 1 μM with a final volume of 2.5 mL. Cells were treated with AuPhos-89 for time periods of 24 h, 48 h, and 72 h. After the desired treatment period, the medium was removed and added to a 15 mL Falcon tube. The wells were washed with 5 mL of PBS and added to the Falcon tube. The cells were trypsinized (1 mL) and added 5 mL of fresh DMEM. All media were combined, and the tube centrifuged at 2000 rpm for 5 minutes to collect the pellet. The medium was decanted, and the pellet suspended in 1 mL of PBS, which was then transferred to a 1 mL Eppendorf tube, centrifuged at 2000 rpm for 5 minutes and suspended in 70% EtOH/PBS solution. These solutions were stored at 4° C. until ready for analysis. Once all treatments had been collected, the cells were collected by centrifuging at 2000 rpm for 5 minutes. The cells were washed twice with PBS (1 mL) and suspended in a 50 μL of RNase solution (100 μg/mL) and 200 μL of a 50 μg/mL PI solution. The solutions were then filtered through a 5 mL polystyrene round-bottom tube fit with a cell-strainer cap. The samples were then analyzed with FACS.

### Example 13: Apoptosis Analysis

MDA-MB-231 cells were seeded at a density of 5×10⁵ cells/well in a 6-well clear bottom plate with a final media volume of 2 mL. The cells were allowed to adhere overnight at 37° C. A stock of AuPhos-89 was prepared fresh in DMSO/DMEM (% v/v=20:80) and added to the desired well at a concentration of 1 μM with a final volume of 2.5 mL and incubated for 15 h at 37° C. A stock of H2O2 was prepared in PBS and the cells treated at a final concentration of 2 mM for 3 hours and used as positive control. When ready for analysis, medium was removed and the wells washed with 5 mL of PBS. The cells were trypsinized (1 mL), 5 mL of DMEM were added to each well, and total volume collected and centrifuged to pellet the cells. The cells were resuspended in 2 mL of fresh media, counted, and reconstituted to a concentration of 1×10⁵ cells/mL. The cells were centrifuged again, and the pellet suspended in 500 μL of Annexin binding buffer. To each sample was added 5 μl of Annexin V-FITC and 5 μl PI and incubated in the dark at room temperature for 5 minutes. The samples were then subjected to FACS analysis.

### Example 14: TMT-Based Quantification Analytical Data

MDA-MB-231 cells were seeded on a petri dish (100 mm×15 mm) and allowed to grow to 85% confluency. The cells were then treated with AuPhos-89 at a concentration of 1 μM for 12 h at 37° C. Cells were harvested, and 1×10⁷ cells were collected. Cell pellets were lysed using 200 ul of RIPA lysis buffer, including protease inhibitors, and centrifuged at 12000 rpm for 15 min at 4° C. And the supernatant was transferred to a new EP tube.

Protein concentration was determined using a BCA kit. Transfer 200 μL sample into anew microcentrifuge tube, to each sample tube, reduced by 10 mM TCEP at 56° C. for 1 h. Alkylated by 20 mM IAA at room temperature in the dark for 1 h, add free trypsin into the protein solution at a ratio of 1:50, and the solution was incubated at 37° C. overnight. The extracted peptides were lyophilized to near dryness. Re-dissolve the sample with 100 mM TEAB. Immediately before use, equilibrate the TMT Label Reagents to room temperature. Add 20 μL of anhydrous acetonitrile to each tube, and allow the reagent to dissolve for 5 minutes with occasional vortexing. Briefly centrifuge the tube to gather the solution. Transfer the samples to the TMT Reagent vial and incubate the reaction for 1 hour at room temperature. Add 8 μL of 5% hydroxylamine to the sample and incubate for 15 minutes to quench the reaction. And combine samples at equal amounts in a new microcentrifuge tube.

Fractionation of the labeled peptides with 6 components using HPLC. The LC and MS used are as follows: Nanoflow UPLC: Ultimate 3000 nano UHPLC system (ThermoFisher Scientific, USA), Q Exactive HF mass spectrometer (Thermo Fisher Scientific, USA): Spray voltage: 2.2 kV, Capillary temperature: 270° C., MS parameters: MS resolution: 120000 at 200 m/z, MS precursor m/z range: 300.0-1650.0. The 6 raw MS files were analyzed and searched against the HUMAN protein database based on the samples' species using Maxquant (1.5.6.5). The parameters were set as follows: the protein modifications were carbamidomethylation (C) (fixed), oxidation (M) (variable); the enzyme specificity was set to trypsin; the maximum missed cleavages were set to 2; the precursor ion mass tolerance was set to 10 ppm, and MS/MS tolerance was 0.6 Da. Only high confident identified peptides were chosen for downstream protein identification analysis. In total, 2052 proteins were identified for this project. Proteins of relative quantitation were divided into two categories. A quantitative ratio over 1.5 was considered up-regulation, while a quantitative ratio of less than 1/1.5 was considered as down-regulation.

### Example 15: Animals

Female, 5 week-old BALB/c mice were purchased from Charles River Laboratories (Wilmington, MA). All mice were quarantined for 1 week prior to use and kept in micro-isolator cages (four mice per cage) in a temperature- and humidity-controlled environment as per the Division of Laboratory Animal Research (DLAR) of University of Kentucky. All mice were maintained in a pathogen-free environment under the care of DLAR of University of Kentucky. The study was performed in compliance with the NIH guidelines (NIH Publication No. 85-23 Rev. 1985) for the care and use of laboratory animals and all experimental procedures were monitored and approved by the Institutional Animal Care and Use Committee (IACUC) of University of Kentucky (USA).

### Example 16: In Vivo Experiment of AuPhos-89

10 female BALB/c mice (5 weeks) were received from Charles River Laboratories (Wilmington, MA), and they had an acclimation period of one week before implanted with 1,000,000 4T1 cells subcutaneously on their flanks. Three days post-implantation, the mice were administered 10 mg/kg AuPhos-89 retro-orbital intravenously (IV), 0.1 mL/mouse formulated as 1% DMSO, 10% Kolliphor, and 89% PBS. The control group was injected with a PBS solution containing 1% DMSO and 1% Kolliphor. AuPhos-89 injection, tumor-size/body-weighing measurement were performed three days a week, and mice were euthanized 19 days later. (n=5 for AuPhos-89, and n=5 for vehicle control)

### Example 17: In Vivo Comparative Experiment of AuPhos-89 and Cisplatin

Mice were implanted with 2,000,000 4T1 cells subcutaneously on their flanks. Five days post-implantation, the mice were administered 10 mg/kg AuPhos-89 and 3 mg/kg cisplatin intraperitoneally (IP). For AuPhos-89, 0.2 mL/mouse was formulated as 1.5% DMSO, 12% Kolliphor, and 86.5% PBS, and cisplatin was dissolved in 100% PBS. The control group was injected with a PBS solution containing 1.5% DMSO and 12% Kolliphor. Injection and tumor-size/body-weight measurement was performed three days a week. (n=5 for AuPhos-89, n=3 for cisplatin, and n=4 for vehicle control).

### Example 18: Hematoxylin and Eosin (H&E) Staining

The mice used in the in vivo comparative experiment of AuPhos-89 and cisplatin were sacrificed at day 14 post tumor cells(4T1) injection. Mice organs (heart, lung, liver, kidney, spleen, and tumor) were fixed in freshly prepared paraformaldehyde (4% in PBS), the fixation time was 24 hours. And those processed for paraffin sectioning. The organs sections of 5 μm were stained with H&E staining and used for histological examination of the organs and tumor. A total of 5 sections per tissues (spanning the full depth of the organ) were examined and photographed using a Nikon Eclipse 55i microscope.

### Example 19: Tissue Biodistribution of AuPhos-89

With four mice inoculated with 4T1 (1,000,000 cells), after 20 days, AuPhos-89 was inserted into mice by IV administration (0.1 mL, 10 mg/kg). Each tissue was obtained by sacrificing the mice (n=2) 1 and 24 hours later. The thus obtained tissues were boiled for 5 hours at 60° C. with 70% nitric acid (0.5 ml) and then boiled again at 60° C. for 10 minutes by adding 35% hydrogen peroxide (0.5 ml). The solution turned yellow and diluted as needed to measure the gold content using a Graphite Furnace Atomic Absorption Spectrometer. Before measuring all samples, the standard solution curves were measured.

### Example 20: Anticancer Compound Design, Synthesis, and Characterization

The organogold(III) bearing bisphosphine ligands, referred to herein as the AuPhos class of compounds, were synthesized as part of efforts to generate diverse gold compounds for biological evaluation. It was rationalized that stabilizing the gold(III) center by efficient ligand tuning will influence biological reactivity and function. To minimize premature deactivation and degradation of anticancer agents, in vivo, it is imperative to enhance their kinetic stability. The use of strongly donating phosphorus containing ligands coupled with (C\hat{ }N)-cyclometalation provides a unique balance between compound stability and efficacy. Optimizing ligands around the gold center decorated with (C\hat{ }N)-cyclometalation has been hampered by the rapid reductive elimination induced by donor ligands to form C(sp²)—X bonds. To resolve this, bisphosphine coupled with mild reaction conditions were used, which minimizes reductive elimination but gives rise to the target compound. Diphenylphosphine benzene was selected as the donor ligand of choice and reacted it with a series of cyclometalated gold(III) starting materials for structure activity relationship (SAR) studies.

Briefly, treatment of HAuCl₄ with the respective benzyl/benzoyl pyridine ligands in refluxing water affords the cyclometalated compounds. Ligand substitution reactions with bisphosphine ligands in chloroform at room temperature overnight resulted in organometallic gold(III) bisphosphine compounds, AuPhos (FIG. 1A-1E). It is worth noting that the products of the reaction scheme in FIG. 1A were verified by structural insights obtained by X-ray crystallography data (vide infra). Thus, the Au(III)-bisphosphine studied in this work likely existed as organgold(III)-bisphosphine compounds without Au—N(sp²) coordination or with Au—N(sp²) coordination as in AuPhos-83. Complexes AuPhos-81-AuPhos-89 were characterized by ¹H NMR, ³¹P NMR, ¹³C NMR spectroscopy (data not shown) and high-resolution mass spectrometry (data not shown). The purity of the compounds was confirmed by elemental analysis. The ³¹P NMR spectra of these compounds show two characteristic peaks in the downfield region of >50 ppm. The resonances show a doublet, which is as a result of cis-P—P coupling. The spectroscopic data obtained were validated by HRMS (ESI) for all compounds, showing a characteristic [M−Cl]⁺ molecular ion peak. The ionization pattern and isotopic distribution confirm the potential coordination of one chloride ligand to the gold center.

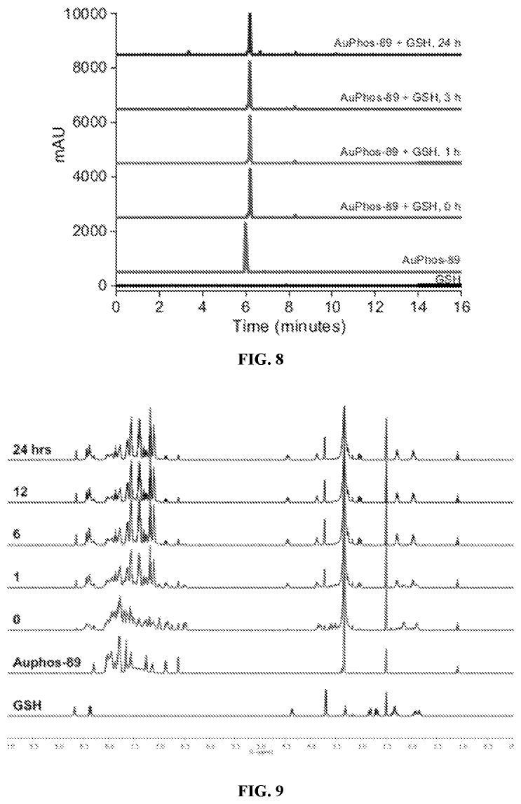

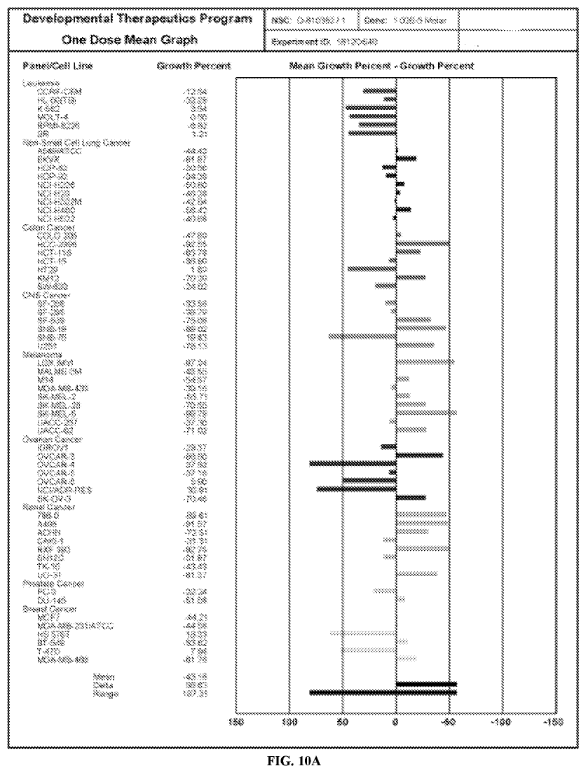

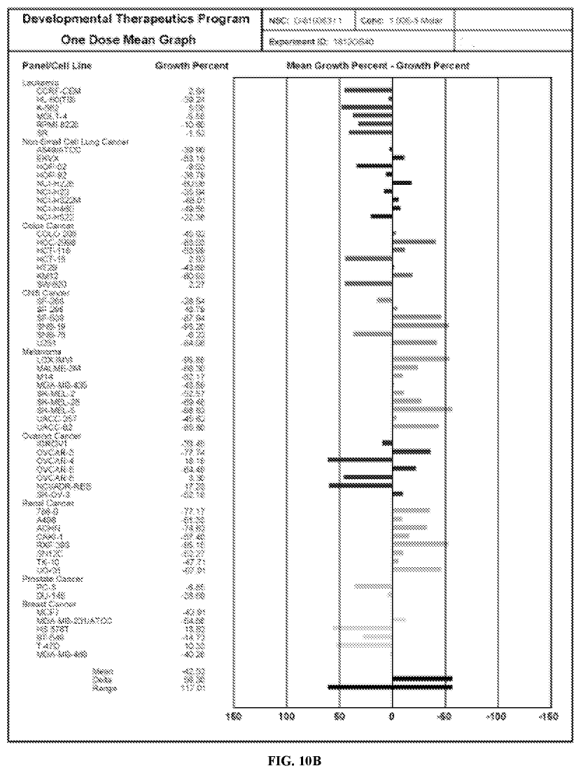

The AuPhos complexes display moisture and air stability. To study the biological stability of AuPhos complexes, reactivity with glutathione (L-GSH), a representative intracellular antioxidant, was evaluated using HPLC in aqueous solution (FIG. 8). Interestingly, the chromatogram of AuPhos-89 in the reaction showed little change over the estimated 24 h period. Similarly, stability studies of AuPhos-89 using ¹H-NMR spectroscopy were performed (FIG. 9). The proton resonances corresponding to AuPhos-89 or L-GSH in the mixed reaction solution of AuPhos-89 and L-GSH in DMSO-d₆ were minimally altered after 24 h. Changes in proton resonances at 0 and 1 h seem to be due to the slow solubility of L-GSH in DMSO solvent. Together, these experiments confirm the stability of AuPhos-89 in the biological environment.

Single crystals of AuPhos-81, 82, and 84 were grown by vapor diffusion of ether into a solution of concentrated gold complex in DMF at 4° C. or room temperature, and AuPhos-83 was crystallized using acetone/THF. The single crystals were analyzed by X-ray diffraction to determine the molecular structures. The crystal structures solved for AuPhos-81, 82 and 84 reveal a four-coordinate structure with the carbon from the arylpyridine bonded to the gold atom, chloride and diphosphine coordinated to the gold atom in a square-planar geometry. Based on the crystal structure of AuPhos-83, a cyclometalated compound is obtained, where the arylpyridine ring is bonded through the carbon and nitrogen atoms to the gold. The Au—N bond length is 2.096 (4), indicative of a strong bond. The organogold character is defined by the existence of the Au—C bond with a bond length within the range of 2.072 (8)-2.0819 (19) for AuPhos-81, 82 and 84. In contrast, the Au—C bond for AuPhos-83 is slightly longer at 2.100 (5). Another interesting feature of the crystal structure of AuPhos-83 is the potential axial interaction of the cationic Au center and chloride ligand. Whereas the observation is found in the solid state with a relatively longer bond length of 2.8790 (11), it presents opportunities to investigate the type of interaction or bonding in solution and whether Au(III) systems can accommodate a fifth coordination. The use of the 1,2-bis(diphenylphosphino)benzene (DPPB) ligand provides a rigid backbone for gold chelation. Table 6 shows selected interatomic bond distances and angles of the crystal structure of AuPhos-81, 82, 83, and 84 as displayed in FIG. 1B-E. Further analysis of the P1-Au1-Cl1 angles of AuPhos-81, 82, and 84 shows a close to linear angle of ˜173-178°. Overall, the structures of organogold(III)-bisphosphine complexes have been elucidated.

TABLE 6
Selected interatomic distances (Å) and angles (°)
from the crystal structures shown in FIG. 1B-1E below

| | Gold complexes | | | | |
|---|---|---|---|---|---|
| Bond/angle | AuPhos-81 | AuPhos-82 | AuPhos-83 | AuPhos-84 | |
| Au1—N1 | — | — | 2.096(4) | — | — |
| Au1—C1 | 2.0819(19) | 2.074(3) | 2.100(5) | 2.079(8) | 2.072(8) |
| Au1—P1 | 2.2808(5) | 2.2843(7) | 2.3396(12) | 2.289(2) | 2.283(2) |
| Au1—P2 | 2.3602(5) | 2.3451(7) | 2.3406(12) | 2.341(2) | 2.344(2) |
| Au1—Cl1 | 2.3387(5) | 2.3354(6) | 2.8790(11) | 2.342(2) | 2.341(2) |
| P1—C24/C25 | 1.808(2) | 1.811(3) | 1.797(4) | 1.807(8) | 1.804(8) |
| P1—C12/C13 | 1.8180(19) | 1.815(3) | 1.810(5) | 1.806(9) | 1.814(9) |
| P2—C36/C37 | 1.808(2) | 1.799(3) | 1.801(5) | 1.800(8) | 1.808(8) |
| C1—Au1—P1 | 91.10(6) | 93.46(7) | 96.22(14) | 88.7(2) | 178.63(7) |
| N1—Au1—P2 | — | — | 96.46(13) | — | — |
| C1—Au1—P2 | 172.04(6) | 175.92(7) | 175.16(15) | 175.3(2) | 175.5(2) |
| P1—Au1—P2 | 84.459(17) | 82.91(2) | 82.76(4) | 86.55(8) | 86.57(8) |
| N1—Au1—Cl1 | — | — | 96.05(14) | — | — |
| C1—Au1—Cl1 | 89.76(6) | 90.44(7) | 97.65(14) | 90.5(2) | 90.5(2) |
| P1—Au1—Cl1 | 176.814(18) | 173.47(2) | 88.58(4) | 178.91(8) | 178.63(7) |
| P2—Au1—Cl1 | 94.331(17) | 93.01(2) | 87.07(4) | 94.15(8) | 93.79(8) |

### Example 21: NCI-60 Screening

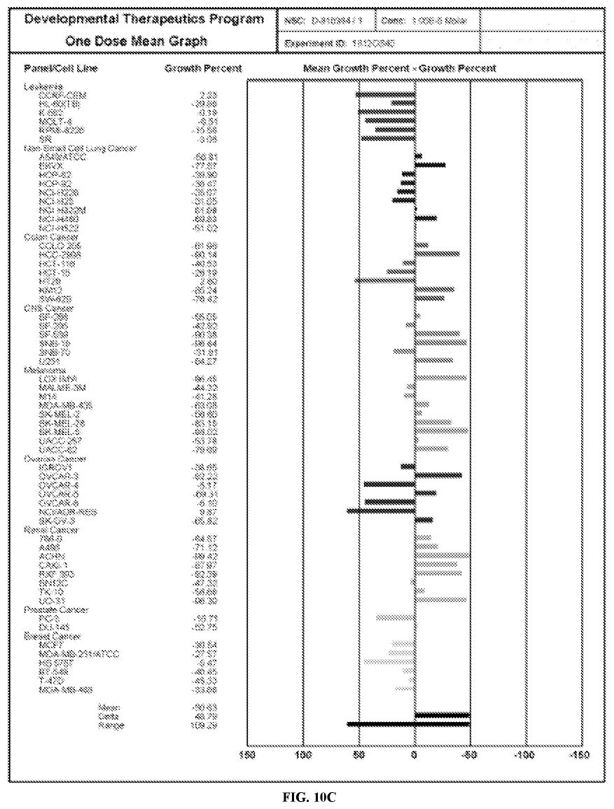

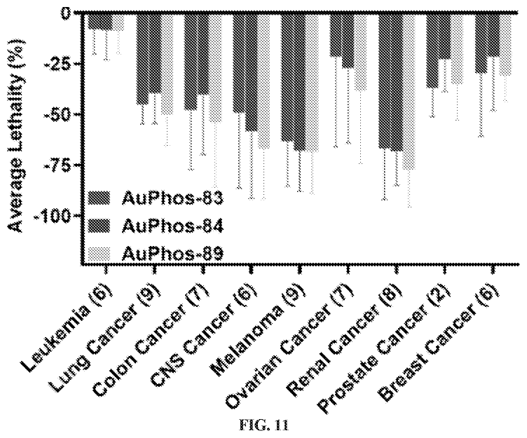

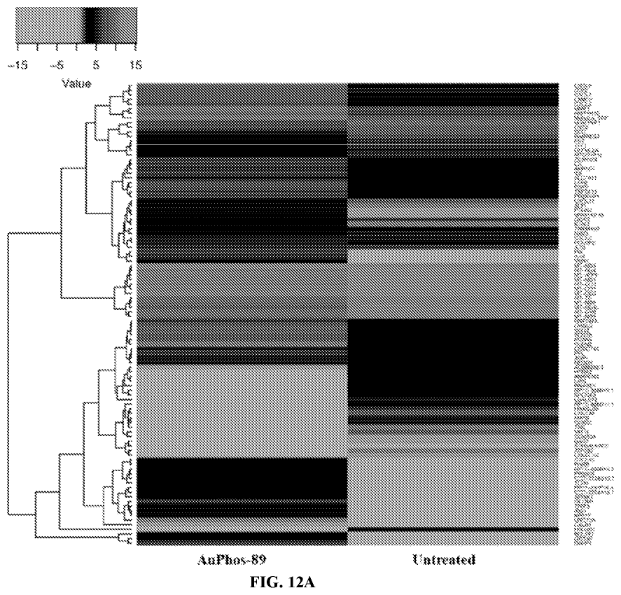

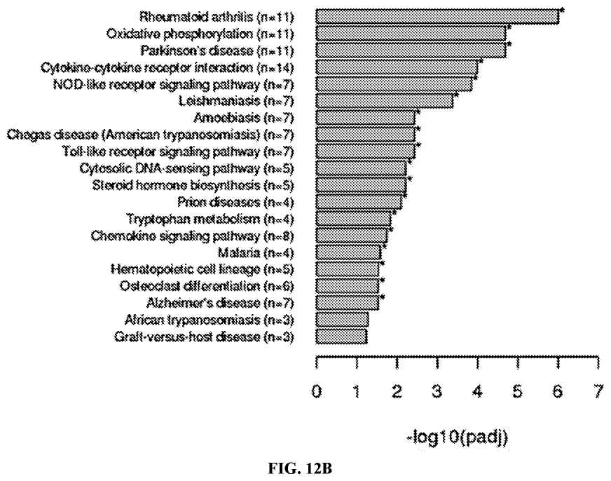

Encouraged by the anti-proliferative potency of the compounds (Table 1 and FIG. 2-6), the cell lines used were expanded and studies were planned to seek insight into the mechanism of action (MOA). Comparative profiling and recent advances in omics technology have expanded the toolkit for target identification and mechanism of action of bioactive molecules. A phenotypic comparison of compounds with known mechanisms can reveal the MOA of compounds with unknown mechanisms, particularly, metal-based compounds. The use of comparative profiling approaches relies on correlating phenotypes obtained with an unknown compound to those within a set of reference compounds with known MOA. It is inferred that compounds with similar MOA will exhibit similar phenotypes. In 1990, the National Cancer Institute compiled a reference set of 60 cancer cell lines (NCI-60) and profiled the sensitivity of each cell line to a large panel of compounds.^{75,76} This has yielded large-scale datasets that are rich resources for the MOA of unknown compounds and target identification. The comparative profiles of three of the AuPhos compounds (AuPhos-83, 84, and 89) were assessed in the NCI-60 screening service. Following a single dose screening at 10 μM, the Developmental Therapeutic Program prioritized the compounds for 5-point dose response assays in the NCI-60 panel. The results reveal that AuPhos-83, 84 and 89 display profound lethality across all cancer types including breast cancer with GI₅₀ in the range of 150 nM-700 nM. All three compounds (AuPhos-83, 84, and 89) showed excellent results across the NCI-60 panel with unbiased lethal effects, especially in central nervous system (CNS) cancer, melanoma, and renal cancers. Surprisingly, the lethal effect on liquid tumors such as leukemia was relatively diminished compared to the solid tumors in the NCI-60 panel. Thus, the compound with superior cell growth inhibitory ability across all cell lines (i.e. AuPhos-89) was prioritized for further biological experiments. These results are shown in the ESI (FIGS. 10A-10C) and summarized in FIG. 11.

Additionally, the COMPARE algorithm, which is a pattern recognition algorithm, can be used to suggest a putative mechanism or the uniquely distinct mechanism of a given agent. Pearson (PCC) and Spearman correlations (SC) are used to estimate the degree of similarity of a test compound, in this case AuPhos, to compounds with known mechanisms in the NCI-60 database. An FDA-approved and investigational compound library from the Cancer Chemotherapy National Service Center (NSC) was used for comparison. The rationale for this compound set was the well-characterized MOA of approved drugs. The AuPhos compounds screened in the NCI-60 panel had no reliable correlation with the drug set used. The filtering conditions utilized GI50, LC50, and TGI derived from the NCI-60 results obtained for AuPhos. Taken together, the comparative profiling of the novel class of gold compounds revealed a different mechanism of action from known drugs, which presents new opportunities for efficacious drugs.

### Example 22: Differential Gene Expression and Biological Pathway Analysis

Based on the exceptional potency of the AuPhos class of compounds and different mechanism of action as derived from the NCI-60 screening, a systems biology approach was used to assist in the quest to unravel the mechanism of action or validate findings from comparative profiling. RNA sequencing was used to determine differential gene expression in MDA-MB-231 cells in response to treatment with 1 μM of AuPhos-89 for 12 h. Briefly, MDA-MB-231 cells were exposed to AuPhos-89 and following the isolation of highly pure RNA, RNA-sequencing was performed for both control- and AuPhos-treated cells with ˜30 million 50-bp paired-end reads generated per sample using the Illumina Hi-seq. Sequence reads were mapped to the hg19 (GRCh37) human genome. 922 DEG was found with 83 upregulated and 94 downregulated genes in response to AuPhos (FIG. 12A). Kyoto encyclopaedia of genes and genomes (KEGG)⁷⁷⁻⁷⁹ (FIG. 12B) pathway analysis uncovered potential processes perturbed by AuPhos-89. The pathway analysis software employed is an extensive library database capable of integrating chemical and biological pathway perturbation processes and is well suited for drug development studies. Differentially expressed genes that contributed to the top five pathways in response to AuPhos-89 included rheumatoid arthritis, oxidative phosphorylation, Parkinson's disease, cytokine-cytokine receptor interaction, and NOD-like receptor signaling. Interestingly, these processes are related to inflammation, which is chiefly regulated by the mitochondria and linked to metabolism. Several differentially expressed genes were related to the electron transport chain, the driver of oxidative phosphorylation. Thus, it was contemplated that AuPhos-89 induces cell death by modulating oxidative phosphorylation and redox pathways in breast cancer cells. Recent findings suggest OXPHOS as a viable therapeutic target in cancer including breast cancer. This led us to investigate the effect of AuPhos-89 on mitochondrial metabolism.

### Example 23: Cellular and Mitochondrial Uptake of AuPhos

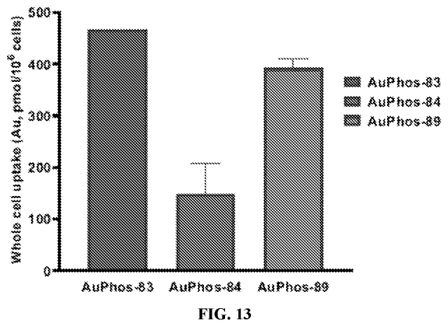

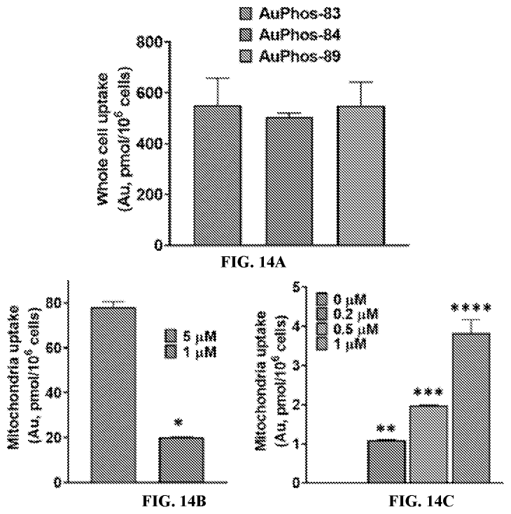

In efforts to deepen the insight into the mechanism of action of AuPhos, the intracellular accumulation was examined. Cell permeability is an essential physicochemical property of drug-like molecules or chemical probes. The ability of compounds to cross the cell membrane to induce cytotoxic effects or engage their targets contributes to their efficacy. To study the intracellular uptake for AuPhos, whole-cell uptake experiments were performed in OVCAR8 cells using AuPhos-83, 84 and 89 and found that an appreciable amount of compound was detected in cells after 15 h (FIG. 13). Compound uptake was confirmed by measuring gold accumulation using graphite furnace atomic absorption spectroscopy (GF-AAS). Given that the preliminary targets identified via NCI-60 screening and RNA-seq were localized in the mitochondria, quantification of AuPhos in mitochondria is imperative. Compound uptake was further assessed in mitochondria organelle fractions of MDA-MB-231 cells after incubation with AuPhos-89 (FIG. 14A-14C). This was to confirm mitochondria localization given the dominant oxidative phosphorylation pathway identified. The gold content measured by GF-AAS showed that the gold compound taken up in the mitochondria of cancer cells is significantly higher (>5-fold) than normal cell mitochondria (FIG. 14B-14C). This could be attributed to the high mitochondrial membrane potential in cancer cells compared to normal cells. Additionally, gold uptake by the mitochondria of MDA-MB-231 compared to NCM460 demonstrates the selective accumulation of the compound for cancerous cells in comparison to normal cells. The high degree of cancer cell selectivity is driven by the upregulation of vitamin and fatty acid transporters in cancer cells. In addition, the lipophilic cationic character of AuPhos facilitates mitochondrial uptake. Together, these findings suggest that AuPhos-89 is taken up into intracellular locations of cancer cells, specifically into the mitochondria.

### Example 24: Role of AuPhos in Enhancing Mitochondrial Respiration

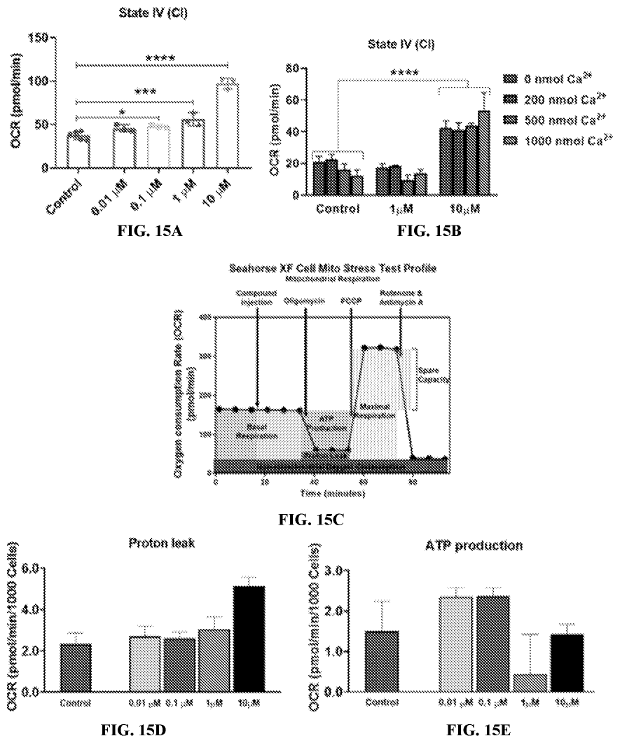

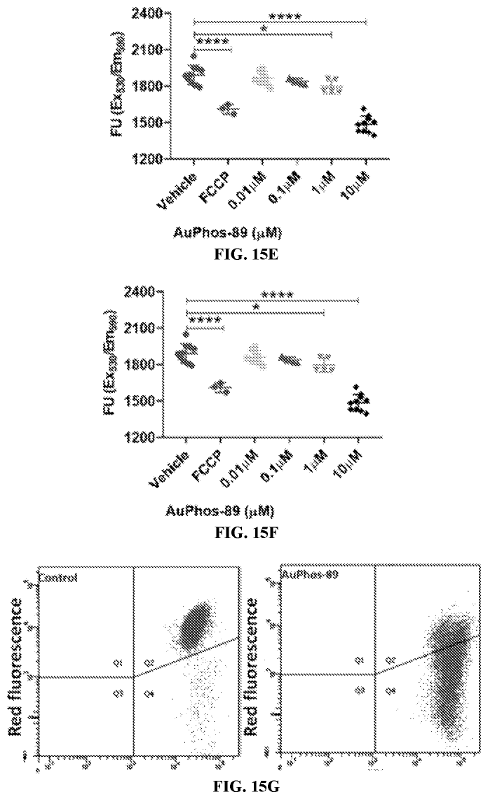

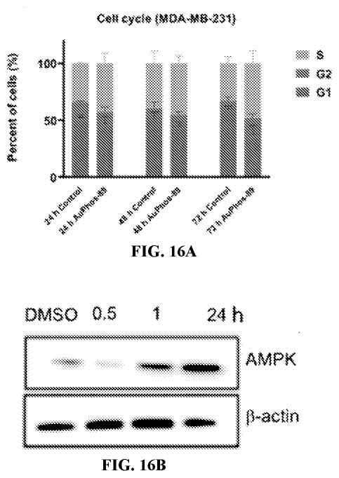

The direct interaction and effect of AuPhos-89 on mitochondria was investigated to assess mitochondrial bioenergetics. A characteristic hallmark of mitochondrial uncoupling activity is the increase of mitochondrial oxygen consumption rate (OCR), even in the presence of F₀F1 ATP synthase inhibitors including oligomycin. Using isolated mitochondria from the liver of C57BL/6J mice, AuPhos-89 at a concentration of 1 μM increased oxygen consumption in the presence of oligomycin (FIG. 15A) as determined using a Seahorse XF analyzer. This phenomenon was dose-dependent up to about 10 μM. Moreover, the induction of OCR was independent of Ca²⁺ (FIG. 15B). Furthermore, at the same concentrations, AuPhos-89 acutely induced proton leak in live MDA-MB-231 cells (FIG. 15D) with diminished ATP production (FIG. 15E). Assessment of the mitochondrial membrane potential (MMP) indicated that there was depolarization of the MMP of MDA-MB-231 cells using the TMRE (FIG. 15F) or JC-1 assay at short compound exposure periods (FIG. 15G). The results support the mitochondrial OXPHOS as a potential target of AuPhos-89.

### Example 25: Cellular Responses Evoked by AuPhos

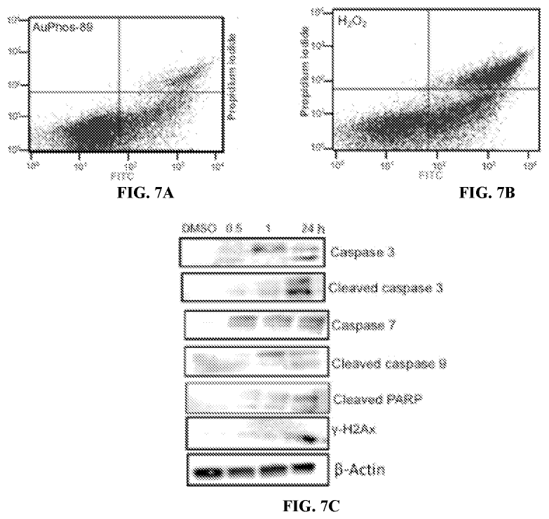

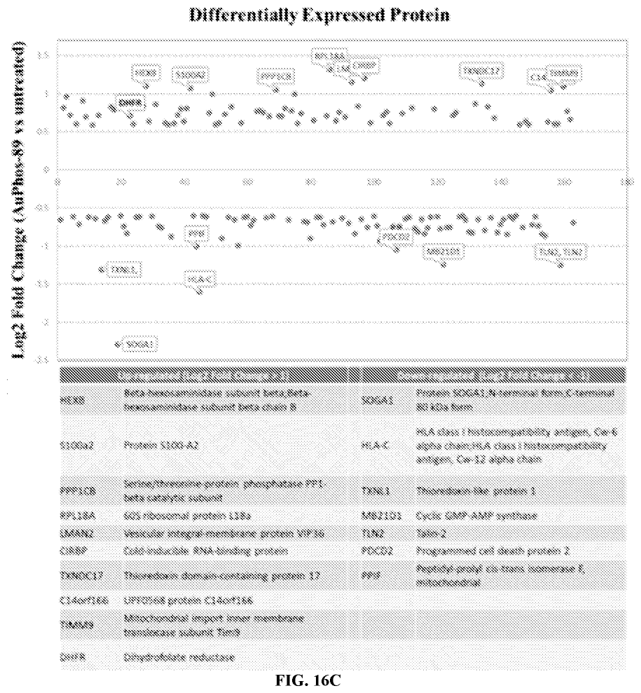

Analysis of cell population in the different phases of the cell cycle revealed no significant changes to the cell cycle, indicating that the compound does not stall cells in these phases under the treatment condition used (FIG. 16A). The DNA binding agent, cisplatin, is known to induce S and G2/M cell cycle arrest in a number of cell lines, supporting its mechanism of DNA cross-linking. When MDA-MB-231 cells were exposed to AuPhos-89 at 1 μM for 15 h, a significant population (˜40%) of cells were in early to late apoptosis as detected by FACS (FIG. 7A). As expected, the control treated with hydrogen peroxide showed significant apoptosis (FIG. 7B). The results suggest that AuPhos induces significant apoptosis as a mode of cell death in breast cancer cells. Furthermore, to determine if AuPhos-89 induces apoptosis through the caspase-mediated apoptotic pathway, western blotting was performed to assess the induction of caspases and cleaved PARP (FIG. 7C). AuPhos-89 was found to activate cleaved caspases, PARP and the cellular energy regulator, AMPK (FIG. 16B), a hallmark of apoptosis. Global changes in proteins were investigated using quantitative proteomics after 12 h of exposing MDA-MB-231 cells to AuPhos-89 at 1 μM. In this tandem mass tag (TMT)-based quantification, protein was extracted from cryo-preserved cell pellets and following tryptic digestion, TMT-labeling was performed. After fractionation, tandem LC-MS/MS was conducted and database search against HUMAN protein database was carried out. Proteins of relative quantification were divided into two categories. A quantitative ratio over 1.5 was considered upregulation while a quantitative ratio less than 1/1.5 was considered as downregulation. A total of 163 differentially expressed proteins were found, 75 of which were upregulated and 88 downregulated (FIG. 16C). Additionally, protein-related mitochondrial metabolism including ADP-ribosylation 4, glutathione-S-transferase, thioredoxin, and enzymes in the glucose pathway was upregulated to compensate for energy deprivation by mitochondrial stress. Overall, the proteomics study supports the other profiling studies described in this work and clearly lays out a systematic pipeline to study new metal-based agents and elucidate their mode of action in an unbiased way.

### Example 26: In Vivo Anticancer Potential of AuPhos-89

Despite the augmented in vitro potency and the novel mechanistic activities reported with other experimental metal-based anticancer agents, many of these compounds have been limited by lack of preclinical evaluation in animal models.

The relative in vivo antitumor efficacy of AuPhos-89 was evaluated as compared to vehicle control. A murine 4T1 cell-line syngeneic of the TNBC model was employed, and AuPhos-89 was administered by 3 weekly IV injections at a dose equivalent of 10 mg kg⁻¹ (n=5 mice per treatment group). A separate group of mice was administered equal volumes of vehicle control at the same time points and via the analogous route. During the first three days after tumor cell implantation and prior to treatment, all mice exhibited equivalent rates of tumor growth. After the first injection, the growth rate of the AuPhos-89 treated group decreased significantly, and this trend was maintained until the end of the experiment (FIG. 7A).

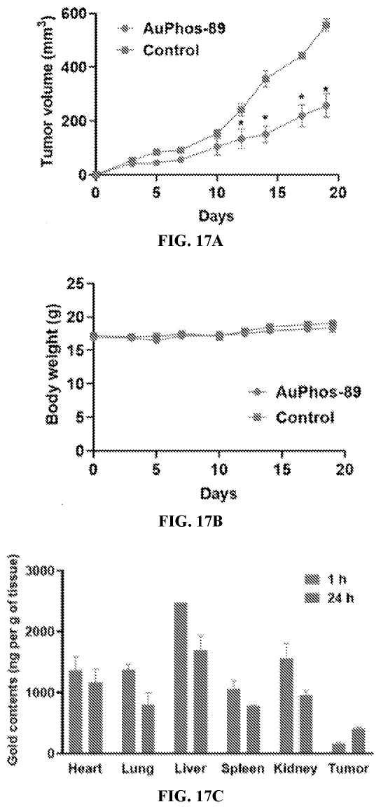

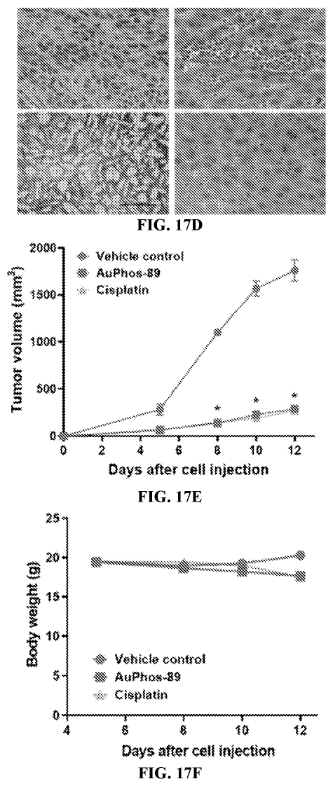

A separate experiment was conducted to compare the antitumor efficacy of the gold compound, AuPhos-89, with that of the FDA approved platinum drug, cisplatin, which is used to treat TNBC. AuPhos-89 (10 mg kg⁻¹) was injected by the intraperitoneal route, and cisplatin was administered at a low concentration of 3 mg kg⁻¹ via intraperitoneal injection due to the toxicity of the drug. Compared with the control group, significant tumor inhibition was observed in the two treatment groups. The weight was kept constant in all the control groups and the experimental group (FIG. 17A-17F).

Tissue biodistribution of therapeutic agents can shed light on drug accumulation and clearance in various organs within test animals. Biodistribution contributes to validating a drug candidate for clinical use. To determine the in vivo biodistribution profile and to validate the effective delivery of the gold compound, AuPhos-89 was injected intravenously at 10 mg kg⁻¹ into female BALB/c mice. After respective time points of 1 h and 24 h, mice were euthanized. Organs including heart, lung, liver, spleen, kidney, and tumor were excised and the gold concentration was analyzed by GF-AAS. Furthermore, the relative quantification of Au levels (as measured by GF-AAS) in the different organs confirmed the uptake of AuPhos-89 that had been introduced into separate mice via IV administration (FIG. 17B). These data give preliminary indication that AuPhos-89 may travel in circulation to reach various organs. Further studies to determine metabolized or degraded gold compounds in circulation using detailed LC-MS/MS are underway.

Together, the data suggest that AuPhos-89 induces significant tumor inhibition in 4T1 tumor bearing mice. Importantly, TNBC is an aggressive form of breast cancer with limited treatment options. Thus, therapeutic agents with different mechanisms of action could provide therapeutic benefit to patients. At the end of the observation period, mice in the AuPhos-89 treatment arm did not exhibit significant change in their body weights, and histological tissue evaluation after H&E staining of excised tumor tissue shows high cellularity in the treated group compared to vehicle control (FIG. 17D). Additionally, signs of liver metastasis were observed in control tissue, suggesting that AuPhos-89 can inhibit liver metastasis associated with 4T1 tumors. Overall, AuPhos shows promise as a potent anticancer agent in vivo.

### Example 27: Chemical Synthesis of Representative Intermediate and Final Compounds

### Dichloro(2-benzoylpyridine)gold(III)

In a 30 ml pressure vessel, 2-benzoylpyridine (0.126 g, 0.686 mmol) and HAuCl₄·3H₂O (0.228 g, 0.579 mmol) were dissolved in distilled water (10 mL). The reaction mixture was stirred for 18 h at 130° C. The precipitate was then vacuum filtered and washed with water to afford an off-white solid (0.175 g, 67% yield), which could then be used without further purification. ¹H NMR (400 MHz, DMSO-d6) δ 9.48 (d, J=5.8 Hz, 1H), 8.55 (t, J=7.7 Hz, 1H), 8.36 (d, J=7.8 Hz, 1H), 8.09 (t, J=6.7 Hz, 1H), 7.76 (d, J=7.6 Hz, 1H), 7.69 (dd, J=6.5, 2.8 Hz, 1H), 7.47 (ddd, J=6.0, 3.9, 1.8 Hz, 2H).

### Dichloro(2-benzylpyridine)gold(III)

In a 30 ml pressure vessel, 2-benzylpyridine (0.085 g, 0.500 mmol) and HAuCl₄ 3H₂O (0.197 g, 0.500 mmol) were dissolved in distilled water (10 mL). The reaction mixture was stirred for 7 hours at 130° C. The precipitate was then vacuum filtered and washed with water to afford an off-white solid (0.179 g, 82% yield), which could then be used without further purification. ¹H NMR (400 MHz, DMSO-d6) δ 9.17 (d, J=5.9 Hz, 1H), 8.26 (t, J=7.6 Hz, 1H), 7.99 (d, J=7.7 Hz, 1H), 7.71 (t, J=6.7 Hz, 1H), 7.41 (d, J=8.0 Hz, 1H), 7.24 (d, J=7.0 Hz, 1H), 7.18 (t, J=7.2 Hz, 1H), 7.07 (t, J=7.4 Hz, 1H), 4.62 (d, J=15.2 Hz, 1H), 4.35 (d, J=15.2 Hz, 1H).

### Dichloro(2-phenylpyridine)gold(III)

In a 30 ml pressure vessel, 2-phenylpyridine (0.076 g, 0.492 mmol) and HAuCl₄ 3H₂O (0.194 g, 0.492 mmol) were dissolved in distilled water (10 mL). The reaction mixture was stirred for 22 h at 130° C. The precipitate was then vacuum filtered and washed with water to afford an off-white solid (0.156 g, 75% yield), which could then be used without further purification. ¹H NMR (400 MHz, DMSO-d6) δ 9.53 (d, J=6.0 Hz, 1H), 8.40 (dd, J=6.5, 1.3 Hz, 2H), 8.00-7.94 (m, 1H), 7.85-7.73 (m, 2H), 7.48 (t, J=7.5 Hz, 1H), 7.42-7.33 (m, 1H).

### Dichloro(benzo[h]quinoline)gold(III)

In a 250 ml round bottom flask, benzo[h]quinoline (0.370 g, 2.06 mmol) was dissolved in CH₃CN (15 mL), and HAuCl₄ 3H₂O (0.750 g, 1.90 mmol) in distilled water (15 mL) was added. A yellow precipitate formed. After stirring for 2 h, the precipitate was filtered. And then it was placed in a ceramic mortar and placed in the oven set at 185° C. for 3 days to afford a brown solid (0.610 g, 72% yield), which could then be used without further purification. ¹H NMR (400 MHz, DMSO-d6) δ 9.70 (d, J=5.7 Hz, 1H), 9.00 (d, J=8.2 Hz, 1H), 8.15-8.03 (m, 4H), 7.92 (d, J=7.8 Hz, 1H), 7.74 (t, J=7.9 Hz, 1H).

### Dichloro(2-(p-tolyl)pyridine)gold(III)

In a 30 ml pressure vessel, 2-(p-tolyl)pyridine (0.082 g, 0.485 mmol) and HAuCl₄ 3H₂O (0.191 g, 0.485 mmol) were dissolved in distilled water (10 mL). The reaction mixture was stirred for 26 h at 130° C. The precipitate was then vacuum filtered and washed with water to afford an off-white solid (0.186 g, 88% yield), which could then be used without further purification. ¹H NMR (400 MHz, DMSO-d6) δ 9.49 (d, J=5.9 Hz, 1H), 8.35 (d, J=5.3 Hz, 2H), 7.85 (d, J=7.8 Hz, 1H), 7.72 (dt, J=8.6, 4.1 Hz, 1H), 7.61 (s, 1H), 7.30 (d, J=7.8 Hz, 1H), 2.40 (s, 3H).

### Dichloro(4-(2-pyridyl)benzaldehyde)gold(III)

In a 30 ml pressure vessel, 4-(2-pyridyl)benzaldehyde (0.103 g, 0.560 mmol) and HAuCl₄·3H₂O (0.206 g, 0.523 mmol) were dissolved in distilled water (10 mL). The reaction mixture was stirred for 23 h at 130° C. The precipitate was then vacuum filtered and washed with water to afford an off-white solid (0.165 g, 70% yield), which could then be used without further purification. ¹H NMR (400 MHz, DMSO-d6) δ 10.01 (s, 1H), 9.59-9.53 (m, 1H), 8.54 (d, J=7.9 Hz, 1H), 8.46 (t, J=7.7 Hz, 1H), 8.29 (s, 1H), 8.22 (d, J=7.9 Hz, 1H), 8.00 (d, J=7.8 Hz, 1H), 7.87 (t, J=6.6 Hz, 1H).

### Dichloro(2-phenoxypyridine)gold(III)

In a 30 ml pressure vessel, 2-phenoxypyridin (0.090 g, 0.526 mmol) and HAuCl₄ 3H₂O (0.204 g, 0.517 mmol) were dissolved in distilled water (10 mL). The reaction mixture was stirred for 22 h at 130° C. The precipitate was then vacuum filtered and washed with water to afford a white-pink solid (0.124 g, 55% yield), which could then be used without further purification. ¹H NMR (400 MHz, DMSO-d6) δ 9.06 (d, J=5.6 Hz, 1H), 8.42 (t, J=7.6 Hz, 1H), 7.86 (d, J=8.2 Hz, 1H), 7.69 (t, J=6.5 Hz, 1H), 7.59 (d, J=8.0 Hz, 1H), 7.37 (d, J=9.2 Hz, 2H), 7.23 (t, J=7.0 Hz, 1H).

### Dichloro(2-anilinopyridine)gold(III)

2-bromopyridine (200 mg, 1.266 mmol), aniline (117.86 mg, 1.266 mmol), t-BuONa (364.988 mg, 3.798 mmol), Pd₂(dba)₃ (57.69 mg, 0.063 mmol), and RuPhos (39.415 mg, 0.066 mol) were loaded into a Schlenk tube equipped with a Teflon-coated magnetic stir bar. The mixture was evacuated and back-filled with nitrogen in three cycles. The flask was then placed into a preheated oil bath and stirred for 48 h at 80° C. After completion of reaction, the flask was allowed to cool to room temperature. The mixture was purified by column chromatography gradient 10-20% ethyl-acetate:hexane to afford the desired product (off-white solid, 190.2 mg, 89.1% yield). ¹H NMR (400 MHz, DMSO-d6) δ 8.97 (s, 1H), 8.14 (s, 1H), 7.67 (d, J=7.9 Hz, 2H), 7.55 (t, J=8.7 Hz, 1H), 7.23 (s, 2H), 6.91-6.81 (m, 2H), 6.74 (d, J=6.7 Hz, 1H). ¹³C NMR (101 MHz, DMSO) δ 156.39, 147.69, 142.20, 137.64, 129.02, 120.81, 118.49, 114.67, 111.09.

In a 30 ml pressure vessel, 2-anilinopyridin (0.0946 g, 0.556 mmol) and HAuCl₄·3H₂O (0.2125 g, 0.540 mmol) were dissolved in distilled water (10 mL). The reaction mixture was stirred for 45 minutes at 130° C. The precipitate was then vacuum filtered and washed with water to afford a dark brown solid (0.1634 g, 69% yield), which could then be used without further purification. ¹H NMR (400 MHz, DMSO-d6) δ 10.70 (s, 1H), 8.82 (d, J=6.5 Hz, 1H), 7.97 (t, J=7.7 Hz, 1H), 7.56 (d, J=8.1 Hz, 1H), 7.40 (d, J=8.5 Hz, 1H), 7.26 (t, J=7.4 Hz, 1H), 7.11 (dd, J=17.5, 7.4 Hz, 2H), 7.02 (t, J=7.6 Hz, 1H).

### AuPhos-82: (2-anilinopyridine)[1,2-Bis(diphenylphosphino)benzene]gold(III) Dichloride

Under normal atmospheric conditions, dichloro(2-anilinopyridine)gold(III) (0.082 g, 0.186 mmol) was placed in a 100 mL of round bottom flask and 10 mL of chloroform was added, the solution turned dark brown. 1,2-Bis(diphenylphosphino)benzene (0.085 g, 0.189 mmol) was added, the solution turned dark yellow instantly. The solution was stirred for about 16 minutes. The solution was monitored by TLC in 5% MeOH in CH2Cl2 as an eluent. Separation of compound was achieved via flash chromatography using CombiFlashR Rf+ Lumen with 5:95/MeOH:CH₂Cl₂. ¹H NMR (400 MHz, Chloroform-d) δ 11.63 (s, 1H), 8.39 (d, J=7.7 Hz, 1H), 8.04 (s, 2H), 7.99-7.82 (m, 3H), 7.81-7.73 (m, 4H), 7.70-7.46 (m, 7H), 7.43 (s, J=7.6 Hz, 1H), 7.29 (s, 1H), 7.20-6.99 (m, 5H), 6.86 (s, 2H), 6.70 (s, 2H), 6.48 (dd, J=18.2, 5.8 Hz, 2H), 6.15 (t, J=6.6 Hz, 1H). ¹³C NMR (101 MHz, Chloroform-d) δ 153.70, 147.10, 140.44, 136.76, 136.74, 136.20, 136.15, 136.06, 136.00, 135.59, 135.51, 135.09, 135.06, 135.02, 134.99, 134.89, 134.76, 134.26, 134.24, 134.16, 133.61, 132.70, 129.93, 129.80, 128.60, 124.76, 124.67, 120.19, 117.21, 116.26, 116.22. ³¹P NMR (162 MHz, Chloroform-d) δ 49.16 (d, J=11.3 Hz), 41.93. HRMS (ESI) (m/z): calcd. for C₄₁H₃₃AuClN₂P₂[M−Cl]⁺ 847.1473, found: 847.1469. Anal. Calcd. for C₄₁H₃₃AuCl₂N₂P₂ 1.1H₂O: C, 54.51; H, 3.93; N, 3.1. Found: C, 54.41; H, 3.82; N, 3.08.

### AuPhos-83: (2-phenoxypyridine)[1,2-Bis(diphenylphosphino) benzene]gold(III) Dichloride

Under normal atmospheric conditions, dichloro(2-phenoxypyridine)gold(III) (0.099 g, 0.226 mmol) was placed in a 100 mL round bottom flask and 10 mL of chloroform was added. 1,2-Bis(diphenylphosphino)benzene (0.108 g, 0.242 mmol) was added, the solution turned yellow instantly. The solution was stirred for about 12 minutes. The solution was monitored by TLC in 5% MeOH in CH2Cl₂ as an eluent. Separation of compound was achieved via flash chromatography using CombiFlashR Rf+ Lumen with 5:95/MeOH:CH₂Cl₂. ¹H NMR (400 MHz, Chloroform-d) δ 8.20 (s, 1H), 8.05 (dd, J=12.8, 7.6 Hz, 2H), 7.82 (t, J=9.9 Hz, 3H), 7.75-7.61 (m, 4H), 7.59-7.27 (m, 15H), 7.13 (s, 2H), 6.96 (dt, J=27.9, 6.6 Hz, 2H), 6.81 (t, J=6.8 Hz, 2H), 6.64 (t, J=7.3 Hz, 1H). ¹³C NMR (101 MHz, Chloroform-d) 6163.41, 153.41, 136.09, 136.00, 135.89, 135.58, 135.47, 135.34, 134.56, 134.51, 134.42, 134.33, 134.29, 134.20, 134.13, 133.64, 133.53, 133.07, 132.41, 129.68, 129.57, 129.29, 129.17, 129.05, 128.92, 127.42, 126.80, 126.70, 126.52, 125.98, 124.31, 123.64, 123.14, 122.56, 121.14, 120.91, 120.85, 118.59, 112.25. ³¹P NMR (162 MHz, Chloroform-d) δ 52.90 (d, J=5.3 Hz), 52.63 (d, J=5.0 Hz). HRMS (ESI) (m/z): calcd. for C₄₁H₃₂AuClNOP₂ [M−Cl]⁺ 848.1313, found: 848.1309. Anal. Calcd. for C₄₁H₃₂AuCl₂NOP₂ 0.7H₂O: C, 54.89; H, 3.75; N, 1.56. Found: C, 54.87; H, 3.76; N, 1.58.

### AuPhos-84: (2-benzylpyridine)[1,2-Bis(diphenylphosphino)benzene]gold(III) Dichloride

Under normal atmospheric conditions, dichloro(2-benzylpyridine)gold(III) (0.098 g, 0.224 mmol) was placed in a 100 mL round bottom flask and 10 mL of chloroform was added. 1,2-Bis(diphenylphosphino)benzene (0.109 g, 0.244 mmol) was added, the solution turned yellow instantly. The solution was stirred for about 15 minutes. The solution was monitored by TLC in 5% MeOH in CH2Cl₂ as an eluent. Separation of compound was achieved via flash chromatography using CombiFlashR Rf⁺ Lumen with 5:95/MeOH:CH₂Cl₂. ¹H NMR (400 MHz, Chloroform-d) δ 8.43 (t, J=4.6 Hz, 1H), 8.27 (d, J=7.4 Hz, 1H), 7.93 (dd, J=28.6, 7.7 Hz, 5H), 7.79 (dt, J=14.6, 7.4 Hz, 4H), 7.67 (t, J=7.2 Hz, 1H), 7.64-7.51 (m, 6H), 7.43 (t, J=7.3 Hz, 1H), 7.36 (t, J=7.2 Hz, 1H), 7.25 (s, 4H), 7.02 (t, J=7.2 Hz, 1H), 6.91 (s, 2H), 6.81 (dd, J=13.5, 7.7 Hz, 3H), 6.70 (s, 1H), 6.58 (s, 1H), 4.92 (d, J=14.1 Hz, 1H), 4.08 (d, J=14.7 Hz, 1H). 13C NMR (101 MHz, Chloroform-d) δ 157.55, 149.21, 148.10, 142.44, 136.44, 136.37, 135.87, 135.76, 135.64, 135.57, 135.47, 135.36, 135.30, 135.19, 135.17, 134.79, 134.68, 134.64, 133.82, 133.78, 133.74, 133.71, 133.33, 133.29, 133.24, 133.14, 133.11, 132.75, 132.64, 130.53, 130.47, 130.30, 130.18, 130.15, 130.03, 129.93, 129.80, 129.77, 129.65, 128.18, 128.14, 127.83, 127.75, 125.02, 124.50, 123.79, 123.75, 123.01, 122.35, 118.81, 49.45. ³¹P NMR (162 MHz, Chloroform-d) δ 50.02, 41.06. HRMS (ESI) (m/z): calcd. for C₄₂H₃₄AuClNP₂ [M−Cl]⁺ 846.1520, found: 846.1528. Anal. Calcd. for C₄₂H₃₄AuC₁₂NP₂ 1.95H₂O: C, 54.97; H, 4.16; N, 1.53. Found: C, 54.91; H, 4.22; N, 1.62.

### AuPhos-85: (2-benzoylpyridine)[1,2-Bis(diphenylphosphino)benzene]gold(I) Dichloride

Under normal atmospheric conditions, dichloro(2-benzoylpyridine)gold(III) (0.091 g, 0.202 mmol) was placed in a 100 mL round bottom flask and 10 mL of chloroform was added. 1,2-Bis(diphenylphosphino)benzene (0.095 g, 0.212 mmol) was added, the solution turned yellow instantly. The solution was stirred for about 5 minutes. The solution was monitored by TLC in 5% MeOH in CH₂Cl₂ as an eluent. Separation of compound was achieved via flash chromatography using CombiFlashR Rf⁺ Lumen with 5:95/MeOH:CH₂Cl₂. ¹H NMR (400 MHz, Chloroform-d) δ 8.62 (d, J=3.8 Hz, 1H), 8.16 (t, J=7.3 Hz, 1H), 8.08-7.93 (m, 4H), 7.92-7.77 (m, 3H), 7.64 (dt, J=14.2, 6.5 Hz, 7H), 7.58-7.47 (m, 7H), 7.39 (ddd, J=32.2, 15.0, 7.4 Hz, 4H), 7.26 (s, 1H), 7.16 (dq, J=15.1, 7.5 Hz, 3H), 6.93 (dd, J=14.1, 7.9 Hz, 2H). ¹³C NMR (126 MHz, Chloroform-d) δ 196.44, 196.41, 159.84, 158.81, 155.29, 148.68, 138.71, 138.64, 138.16, 138.13, 137.20, 137.02, 136.94, 136.72, 136.66, 136.40, 136.36, 136.31, 136.29, 135.97, 135.95, 135.85, 135.57, 135.52, 135.46, 135.41, 135.34, 135.02, 135.00, 134.80, 134.70, 134.58, 134.54, 134.51, 134.35, 134.30, 134.28, 134.08, 134.01, 133.98, 133.89, 133.82, 133.73, 133.69, 133.64, 133.61, 133.49, 133.30, 133.21, 132.21, 132.14, 131.38, 130.97, 130.86, 130.26, 130.16, 130.00, 129.35, 129.24, 128.97, 128.88, 128.82, 128.72, 126.37, 126.28, 124.99, 124.29, 124.16, 123.82, 123.70, 122.76, 122.20, 119.80, 119.22. ³¹P NMR (162 MHz, Chloroform-d) δ 55.09 (d, J=7.5 Hz), 53.47 (d, J=7.1 Hz). HRMS (ESI) (m/z): calcd. for C₄₂H₃₂AuClNOP₂ [M−Cl]⁺ 860.1313, found: 860.1321. Anal. Calcd. for C₄₂H₃₂AuCl₂NOP₂ 1.2H₂O: C, 54.84; H, 3.79; N, 1.52. Found: C, 54.94; H, 3.78; N, 1.53.

### AuPhos-86: (benzo[h]quinoline)[1,2-Bis(diphenylphosphino)benzene]gold(III) Dichloride

Under normal atmospheric conditions, dichloro(benzo[h]quinoline)gold(III) (0.080 g, 0.179 mmol) was placed in a 100 mL round bottom flask and 10 mL of chloroform was added, the solution turned dark brown. 1,2-Bis(diphenylphosphino)benzene (0.084 g, 0.188 mmol) was added, the solution turned dark yellow instantly. The solution was stirred for about 22 minutes. The solution was monitored by TLC in 5% MeOH in CH2Cl₂ as an eluent. Separation of compound was achieved via flash chromatography using CombiFlashR Rf+ Lumen with 5:95/MeOH:CH₂Cl₂. ¹H NMR (400 MHz, Chloroform-d) δ 8.15-8.02 (m, 3H), 7.97-7.85 (m, 5H), 7.81 (t, J=8.5 Hz, 1H), 7.68 (dq, J=17.1, 8.7, 7.7 Hz, 10H), 7.41 (dd, J=13.7, 7.9 Hz, 2H), 7.29 (s, 2H), 7.21 (dd, J=7.6, 4.7 Hz, 2H), 7.02 (s, 7H). ¹³C NMR (101 MHz, Chloroform-d) δ 152.98, 151.72, 146.87, 137.72, 137.63, 136.87, 136.61, 136.54, 136.24, 136.21, 136.17, 136.15, 136.08, 135.94, 135.63, 135.58, 134.15, 134.04, 133.96, 133.76, 133.73, 133.67, 133.65, 133.13, 133.01, 130.33, 130.21, 129.29, 129.17, 127.93, 126.98, 126.97, 125.53, 124.93, 121.76. ³¹P NMR (162 MHz, Chloroform-d) δ 52.38 (d, J=11.4 Hz), 51.33 (d, J=11.4 Hz). HRMS (ESI) (m/z): calcd. for C₄₃H₃₂AuClNP₂ [M−Cl]⁺ 856.1364, found: 856.1367. Anal. Calcd. for C₄₃H₃₂AuCl₂NP₂ 1.75H₂O: C, 55.89; H, 3.87; N, 1.52. Found: C, 55.77; H, 3.95; N, 1.64.

### AuPhos-87: (2-(p-tolyl)pyridine)[1,2-Bis(diphenylphosphino)benzene]gold(III) Dichloride

Under normal atmospheric conditions, dichloro(2-(p-tolyl)pyridine)gold(III) (0.084 g, 0.192 mmol) was placed in a 100 mL round bottom flask and 10 mL of chloroform was added. 1,2-Bis(diphenylphosphino)benzene (0.089 g, 0.199 mmol) was added, the solution turned yellow instantly. The solution was stirred for about 14 minutes. The solution was monitored by TLC in 5% MeOH in CH2Cl₂ as an eluent. Separation of compound was achieved via flash chromatography using CombiFlashR Rf+ Lumen with 5:95/MeOH:CH₂Cl₂. ¹H NMR (400 MHz, Chloroform-d) δ 8.13-7.97 (m, 3H), 7.80 (dt, J=17.3, 8.6 Hz, 5H), 7.67 (dd, J=15.5, 6.3 Hz, 6H), 7.58-7.35 (m, 6H), 7.30 (s, 4H), 7.22-7.09 (m, 4H), 7.00 (d, J=7.9 Hz, 1H), 6.79-6.67 (m, 2H), 2.11 (s, 3H). ¹³C NMR (101 MHz, Chloroform-d) δ 147.01, 137.69, 136.43, 136.34, 136.08, 136.06, 135.63, 134.17, 134.14, 134.06, 133.95, 133.68, 133.65, 133.45, 133.34, 130.29, 130.29, 130.18, 129.86, 129.73, 129.28, 129.19, 128.32, 125.38, 124.84, 121.99, 121.28, 120.48, 21.33. 31P NMR (162 MHz, Chloroform-d) δ 52.08 (d, J=12.0 Hz), 51.51 (d, J=12.1 Hz). HRMS (ESI) (m/z): calcd. for C₄₂H₃₄AuClNP₂ [M−Cl]⁺ 846.1520, found: 846.1513. Anal. Calcd. for C₄₂H₃₄AuCl₂NP₂ 1.7H₂O: C, 55.24; H, 4.13; N, 1.53. Found: C, 55.20; H, 4.10; N, 1.59.

### AuPhos-88: (4-(2-pyridyl)benzaldehyde)[1,2-Bis(diphenylphosphino)benzene]gold(III) Dichloride

Under normal atmospheric conditions, dichloro(4-(2-pyridyl)benzaldehyde)gold(III) (0.077 g, 0.172 mmol) was placed in a 100 mL round bottom flask and 10 mL of chloroform was added. 1,2-Bis(diphenylphosphino)benzene (0.082 g, 0.182 mmol) was added, the solution turned pale pink instantly. The solution was stirred for about 13 minutes. The solution was monitored by TLC in 5% MeOH in CH2Cl₂ as an eluent. Separation of compound was achieved via flash chromatography using CombiFlashR Rf+ Lumen with 5:95/MeOH:CH₂Cl₂. ¹H NMR (400 MHz, Chloroform-d) δ 9.78 (s, 1H), 8.05 (d, J=30.8 Hz, 3H), 7.94 (d, J=9.9 Hz, 1H), 7.83 (dd, J=12.3, 7.5 Hz, 6H), 7.77-7.64 (m, 7H), 7.51 (dd, J=15.9, 8.4 Hz, 4H), 7.31 (s, 4H), 7.16 (s, 4H), 6.91 (s, 1H), 6.80 (s, 1H). ¹³C NMR (101 MHz, Chloroform-d) δ 191.32, 155.80, 147.47, 138.14, 136.95, 136.69, 136.62, 136.35, 136.13, 135.99, 135.97, 135.75, 135.70, 134.49, 134.46, 134.09, 133.98, 133.91, 133.88, 133.39, 133.27, 130.38, 130.27, 130.13, 130.00, 129.66, 129.57, 128.80, 125.00, 124.44, 123.52, 122.09, 121.73, 121.04. 31P NMR (162 MHz, Chloroform-d) δ 53.02 (d, J=9.6 Hz), 52.69 (d, J=9.4 Hz). HRMS (ESI) (m/z): calcd. for C₄₂H₃₂AuClNOP₂ [M−Cl]⁺ 860.1313, found: 860.1282. Anal. Calcd. for C₄₂H₃₂AuCl₂NOP₂ 1.4H₂O: C, 54.73; H, 3.81; N, 1.52. Found: C, 54.73; H, 3.85; N, 1.55.

### AuPhos-89: (2-phenylpyridine)[1,2-Bis(diphenylphosphino)benzene]gold(III) Dichloride

Under normal atmospheric conditions, dichloro(2-phenylpyridine)gold(III) (0.152 g, 0.360 mmol) was placed in a 100 mL round bottom flask and 10 mL of chloroform was added, the solution turned brown. 1,2-Bis(diphenylphosphino)benzene (0.170 g, 0.380 mmol) was added, the solution turned yellow instantly. The solution was stirred for about 11 minutes. The solution was monitored by TLC in 5% MeOH in CH₂Cl₂ as an eluent. Separation of compound was achieved via flash chromatography using CombiFlashR Rf⁺ Lumen with 5:95/MeOH:CH₂Cl₂. ¹H NMR (400 MHz, Chloroform-d) δ 8.11-7.95 (m, 3H), 7.78 (d, J=8.7 Hz, 5H), 7.65 (dd, J=17.7, 6.2 Hz, 7H), 7.52-7.34 (m, 5H), 7.21-7.01 (m, 6H), 6.83 (d, J=8.7 Hz, 2H). ¹³C NMR (101 MHz, Chloroform-d) δ 157.23, 147.10, 140.32, 137.78, 136.33, 136.26, 136.06, 136.01, 135.91, 135.17, 135.14, 134.20, 133.99, 133.90, 133.68, 133.33, 133.23, 130.61, 130.52, 130.24, 130.22, 130.15, 130.12, 129.87, 129.75, 129.65, 129.57, 127.41, 125.25, 124.68, 122.42, 121.88, 121.16, 120.86. ³¹P NMR (162 MHz, Chloroform-d) δ 51.94 (d, J=13.0 Hz), 51.84 (d, J=11.3 Hz). HRMS (ESI) (m/z): calcd. for C₄₁H₃₂AuClNP₂ [M−Cl]⁺ 832.1364, found: 832.1365. Anal. Calcd. for C₄₁H₃₂AuCl₂NP₂ 1.7H₂O: C, 54.77; H, 3.97; N, 1.56. Found: C, 54.76; H, 3.99; N, 1.53.

### Example 28: Discussion Related to Examples 1-27

This report details the synthesis of novel gold(III)-bisphosphines that modulate mitochondrial respiration as anticancer agents in vitro and in vivo. Compounds that disrupt mitochondria with good pharmacokinetics and a wide therapeutic window remain an unmet need. Thus, a gold-based platform as a mitochondria respiration modulator holds promise as a useful therapeutic agent in a variety of diseases including cancer. The developed organometallic gold(III) compounds are relatively stable in solution even in the presence of physiologically relevant concentrations of glutathione, a major biological thiol. These complexes are potent across a wide range of cancer cell lines with significant lethality in the NCI-60 cell line with no cross-resistance. The compounds display high cellular uptake in cancer cells of >100 pmol per million cells. A systems biology approach was used to gain insight into the mechanism of action of the prioritized gold agent, AuPhos-89. The global effects of AuPhos on MDA-MB-231 cells identified biological processes related to oxidative phosphorylation, rheumatoid arthritis, Parkinsons disease, cytokine-cytokine receptor interaction, and NOD-like receptor signalling as prominent pathways by KEGG analysis. Using bioenergetics to assess mouse liver mitochondria and liver cancer cells, it was confirmed that AuPhos stimulates OCR leading to oxidative stress and, consequently, cell death via apoptosis. Therapeutic indices including the maximum tolerated dose, tissue biodistribution and efficacy reveal that AuPhos is a promising gold(III) anticancer agent.

### Example 29: Gold(III)-Phosphine Complexes as Mitochondrial OXPHOS Uncouplers

Changing the geometries and stabilizing ligands of gold compounds, such as the use of bidentate frameworks, promotes different mitochondrial effects. A systematic SAR study to develop metal-based mitochondrial modulators does not exist. To address these concerns, unique gold (III) phosphines scaffolds were designed. These compounds possess superior anticancer activity in the nanomolar range in TNBC cells. The ligands employed possess different electronic, steric and lipophilic properties that control the redox potential, stability, solubility, and effect on mitochondria function. The ligands used do not induce cytotoxicity on their own even up to 100 μM.

Based on previous work that described the anticancer activity of highly potent gold(I)/(III)-phosphine compounds, the synthetic protocol was expanded to include C,N-cyclometalated ligands for improved stability in gold(III) anticancer agents. The following is a reactions scheme for synthesis of representative gold (III) compounds:

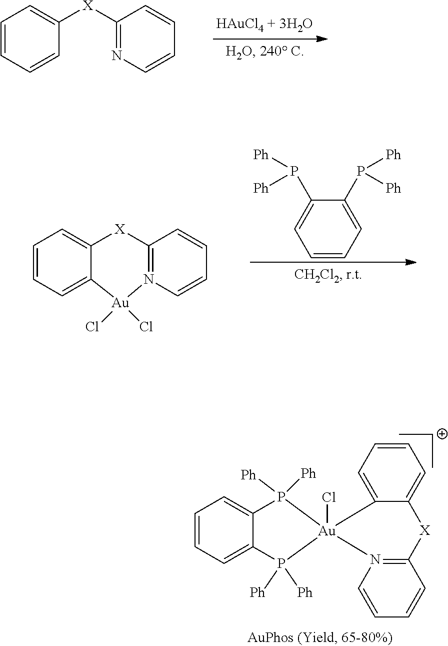

The compounds possess a rare geometry, a square bipyramid, for a gold(III) compound. These compounds overcome the rapid kinetic lability and thiol reactivity in physiological medium observed in previous gold compounds such as auranofin. These compounds also show high cellular uptake and in vitro cytotoxicity in breast cancer cells. To understand the activity of the AuPhos scaffold, select compounds AuPhos 83-89 were compared to a wide range of validated anticancer drugs via the well-established National Cancer Institute's reference panel screen set of 60 tumor cell types (NCI-60). In this screening service, the compound is administered in a single-dose of 10 μM to a range of 60 different cancer cell lines. Following a single-dose testing, the NCI selected compounds AuPhos 83-89 for a 5-dose testing based on a threshold set by the NCI (accession codes: 7-NSC810361; 8-NSC81032; 9-NSC810363).

The results show that the gold compounds are highly potent against the panel of breast cancer cells with GI₅₀ values within ˜150-700 nM. Initial biological investigation into this class of compounds reveals a significant uncoupling effect of mitochondrial OXPHOS with inhibitory effects on cytoskeletal networks responsible for cell migration based on transcriptomics and quantitative proteomics studies. This has implications for the suppression of breast cancer invasion and metastasis⁸⁷. A representative analog of this class shows tumor growth inhibition in both TNBC syngeneic (4T1 in BALB/c) and xenograft (4T1-iRFP in NSG mice), hence, these gold compounds are efficacious in metastatic/aggressive breast cancer mouse models. Interestingly, these compounds are amenable to functionalization through the carbonyl group, either through oxime or reductive amination type modifications and can be targeted to tumors to minimize toxic side effects. These uniquely diverse compounds have a rare square bipyramidal geometry. Biological evaluation of the compounds demonstrates high potency against TNBC in vitro and in vivo with a different mechanism of action from known metal complexes. The syntheses of these compounds are facile and high yielding and will therefore provide a modular platform for ligand tuning with access to a library of potent anticancer agents.

Synthesis and characterization of AuPhos compounds: Under normal atmospheric conditions, in a 25 mL round bottom flask was placed cyclometalated Au(III)Cl₂ (˜63.1 mg, 0.150 mmol). CHCl₃ (10.0 mL) was added and the solution (white suspension) was stirred at room temperature for 1-2 min. To the solution was added 1,2-bis(diphenylphosphino)benzene (68.4 mg, 0.153 mmol). The solution turned pale yellow instantly. The solution was monitored by TLC using 5:95/MeOH:CH₂Cl₂ as an eluent. Compounds were purified via flash chromatography using CombiFlash Rf+ Lumen with 5:95/MeOH:CH₂Cl₂.

In a cytotoxicity experiment using crystal violet assay conducted on MDA-MB-231 breast cancer cells, gold compounds show a robust antiproliferative effect. The measured IC₅₀ values range from a minimum of 0.1957 to a maximum of 0.4099 μM, demonstrating the potent inhibitory effect of gold compounds on breast cancer cells. The toxicity of gold compounds against cancer cells was reconfirmed through NCI-60 screening experiments. As a result of exposing 3-83, 84, and 89 gold compounds with 60 other cancer cells, the average values of all three compounds ranged from −42 to −50 in % lethality.

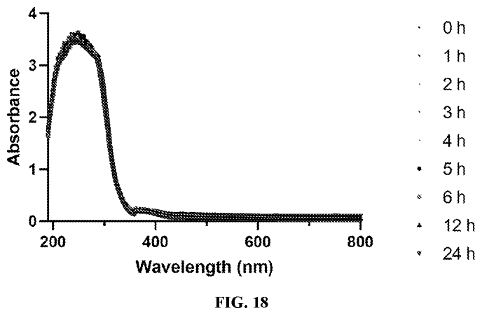

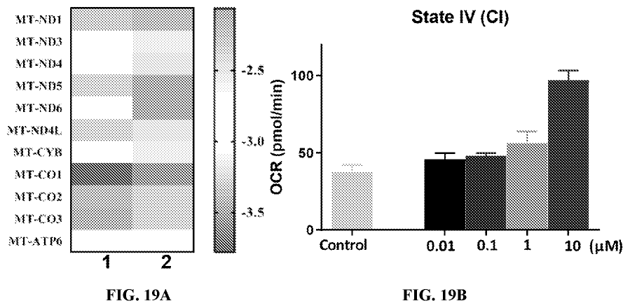

To determine the stability of gold compounds under physiological conditions in the presence of biological nucleophiles, UV absorbance measurements were conducted over a 24 h time period. Briefly, AuPhos was dissolved in media from a DMSO stock. To this solution was added 10 mM L-glutathione. As shown in FIG. 18, no change in the absorption spectra was observed until 24 hours, indicative of high solution stability.

Compounds belonging to the AuPhos class demonstrate IC₅₀s of 150-400 nM in MDA-MB-231 cells. Detailed mitochondrial studies using mouse liver mitochondria and MDA-MB-231 cells support an uncoupling OXPHOS activity by AuPhos in a dose-dependent manner (FIG. 19A-19B). This observation was mitochondrial complex I- and complex II-driven when different substrates, i.e. pyruvate and succinate, were used to measure the oxygen consumption rate. Transcriptomics was used to study global changes in mRNA in response to a 12 h exposure of AuPhos to MDA-MB-231 cells. Analysis of differentially expressed genes reveal that mitochondrial OXPHOS genes: MT-ND1-5, MT-CYB, MT-CO1-3, and MT-ATP6 were modulated. Based on the instant findings, AuPhos uncouples mitochondria leading to reduction of ATP production in MDA-MB-231 cells.

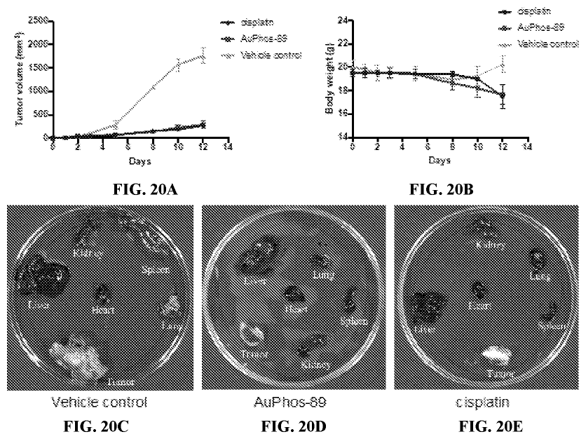

Through structure-activity relationship studies, a functional analog (which was named AuPhos-89, IC₅₀=220 nM) was identified that retained excellent solution stability, enhanced plasma stability and pharmacokinetic properties and was suitable for in vivo studies. Antitumor activity was first assessed by six treatments (thrice a week, intraperitoneally) at 10 mg kg⁻¹ day⁻¹, beginning at 5 mm diameter tumors after subcutaneous implantation of 4T1 cells in BALB/c mice. Mice treated with Au-Phos-89 showed reduced tumor volume and increased rates of survival in comparison to mice treated with the vehicle control (FIG. 20A-20E). Tumor-bearing mice treated with a cisplatin dose of 3 mg/kg (six treatments, thrice a week) showed comparable results to AuPhos-89 treatment with associated toxicity. Murine TNBC 4T1 cells engineered to express RFP were also subcutaneously injected into flanks of immunocompromised mice in the presence of Matrigel. After visible tumors were detected at 3 days, AuPhos-89 was administered at 10 mg kg⁻¹ day⁻¹ (six treatments, thrice a week), which resulted in a significant attenuation of growth compared with controls. (data not shown). Haematoxylin and eosin (H&E) staining indicate reduced cellularity and proliferation in tumors treated with AuPhos-89 compared to vehicle-treated mice. Additionally, liver metastasis was associated with 4T1 tumor-bearing mice. In tumors of control mice, tumor cells were found in the liver of representative H&E sections whereas this was not visible in AuPhos-89-treated mice. Taken together, gold compounds show promise in their use as anticancer agents against TNBC. The proposed studies will enable the identification of improved anticancer drugs based on gold.

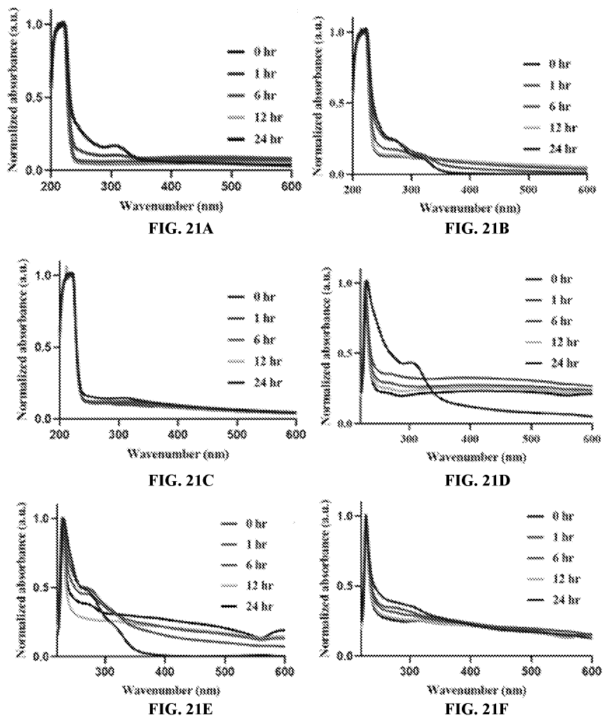

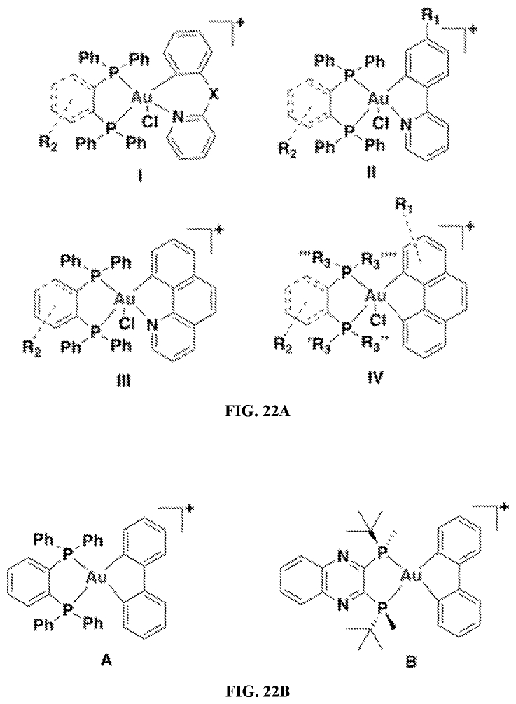

To study the stability of the gold(III) compounds in solution, the UV-vis profile of the representative compounds was evaluated in biologically relevant media at physiological temperature of 37° C. Given the common limitation of gold compounds to promiscuously bind thiol containing biomolecules, the interaction of gold(III) complex AuPhos89 was investigated with physiologically relevant concentration of L-glutathione (10 mM) in Dubelcco modified essential medium (DMEM). There was no alteration to the absorption band of AuPhos-89 in the reaction solution over a period of 24 h at 37° C. Additionally, there were no visible changes in color or brown stains that rule out reduction to elemental gold. Second, since RPMI-1640 contains a lot more thiol reducing agents, solutions of AuPhos-89 in RPMI-1640 were prepared and found no changes to the absorption spectra over 24 h at 37° C. (FIG. 21A-21F).

### Example 30: Structures and Characterization of Compounds

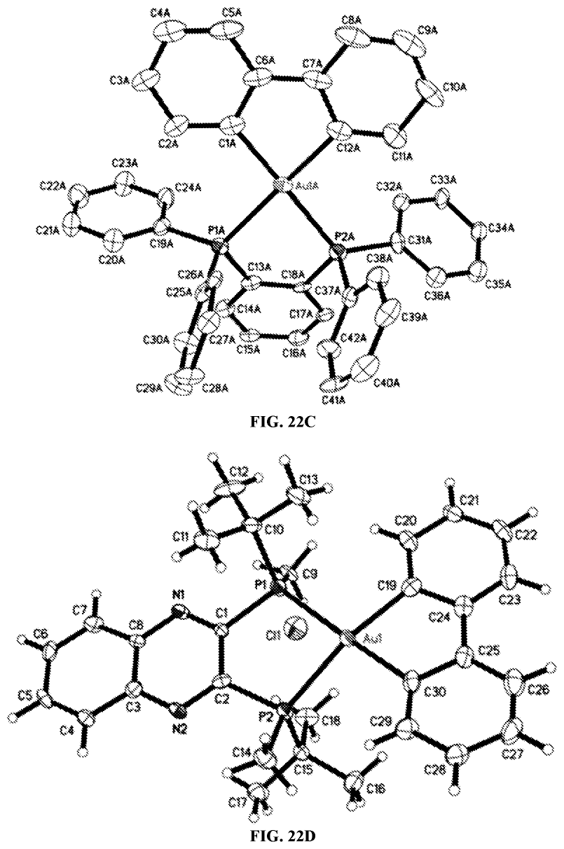

Various compounds are presented in FIG. 22A, with exemplary Compounds A and B presented in FIG. 22B, which are Au^{III} compounds combined with C\hat{ }C ligands and chiral and achiral P\hat{ }P ligands. The compounds were characterized through NMR analysis (data not shown) and X-ray single-crystal structure. Crystal structures for Compound A and Compound B are presented in FIG. 22C and FIG. 22D, respectively.

### Example 31: Anticancer Activity of Compounds

Compounds A and B were studied and determined to exhibit potent anticancer activity. Table 7 includes results of a representative study to determine in vitro antiproliferative activity.

TABLE 7
In vitro antiproliferative activity. IC₅₀
values for B across a panel of cell lines.

| | MDA-MB-231 | MDA-MB-468 | 4T1 | BT-333 |
|---|---|---|---|---|
| B | 0.11 | 0.49 | 0.85 | 0.76 |

IC₅₀ values were determined for B across a panel of cell lines. Cells were seeded at a density of 4000 cells per well and treated for 72 h. IC₅₀ values are presented as the mean±s.e.m (n=3). As shown, in the representative experiment using compound B and four different cancer cell lines, the cytotoxicity of compound B was found to be potent against several cancer cell lines.

### Example 31: Targeting

A targeted form of a Au^{III} compound was developed by linking a representative targeting moiety, a Biotin molecule, to a representative AuPhos compound. The structure of this representative targeted compound is as follows:

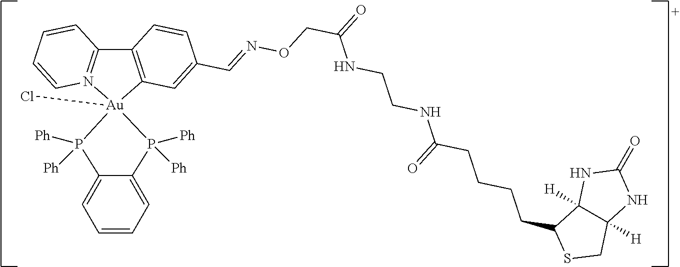

The compound was characterized by ¹H/³¹P NMR spectroscopy and mass spectroscopy (data not shown).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. B. Sirohi, S. Ashley, A. Norton, S. Popat, S. Hughes, P. Papadopoulos, K. Priest and M. O'Brien, Early Response to Platinum-Based First-Line Chemotherapy in Non-small Cell Lung Cancer May Predict Survival, J Thorac. Oncol., 2007, 2, 735-740
2. M. Li, Q. Zhang, P. Fu, P. Li, A. Peng, G. Zhang, X. Song, M. Tan, X. Li, Y. Liu, Y. Wu, S. Fan and C. Wang, Pemetrexed plus platinum as the first-line treatment option for advanced non-small cell lung cancer: a meta-analysis of randomized controlled trials, PLoS One, 2012, 7, e37229
3. V. Brabec and J. Kasparkova, Modifications of DNA by platinum complexes. Relation to resistance of tumors to platinum antitumor drugs. Drug resistance updates: reviews and commentaries in antimicrobial and anticancer chemotherapy, Drug Resist. Updates, 2005, 8, 131-146
4. Z. H. Siddik Cisplatin: mode of cytotoxic action and molecular basis of resistance, Oncogene, 2003, 22, 7265-7279
5. S. R. McWhinney, R. M. Goldberg and H. L. McLeod, Platinum neurotoxicity pharmacogenetics, Mol. Cancer Ther., 2009, 8, 10-16
6. I. Ott and R. Gust, Non platinum metal complexes as anti-cancer drugs, Arch. Pharm., 2007, 340, 117-126
7. Y. Jung and S. J. Lippard, Direct cellular responses to platinum-induced DNA damage, Chem. Rev., 2007, 107, 1387-1407
8. T. Zou, C. T. Lum, C. N. Lok, J. J. Zhang and C. M. Che, Chemical biology of anticancer gold(III) and gold(I) complexes, Chem. Soc. Rev., 2015, 44, 8786-8801
9. K. C. Tong, C. N. Lok, P. K. Wan, D. Hu, Y. M. E. Fung, X. Y. Chang, S. Huang, H. Jiang and C. M. Che, An anticancer gold(III)-activated porphyrin scaffold that covalently modifies protein cysteine thiols, Proc. Natl. Acad. Sci. U.S.A, 2020, 117, 1321-1329
10. J. Maksimoska, L. Feng, K. Harms, C. Yi, J. Kissil, R. Marmorstein and E. Meggers, Targeting large kinase active site with rigid, bulky octahedral ruthenium complexes, J. Am. Chem. Soc., 2008, 130, 15764-15765
11. E. Meggers Targeting proteins with metal complexes, Chem. Commun., 2009, 1001-1010
12. M. Dorr and E. Meggers, Metal complexes as structural templates for targeting proteins, Curr. Opin. Chem. Biol., 2014, 19, 76-81
13. P. Zhang and P. J. Sadler, Redox-Active Metal Complexes for Anticancer Therapy, Eur. J. Inorg. Chem., 2017, 2017, 1541-1548
14. N. Graf and S. J. Lippard, Redox activation of metal-based prodrugs as a strategy for drug delivery, Adv. Drug Delivery Rev., 2012, 64, 993-1004
15. U. Jungwirth, C. R. Kowol, B. K. Keppler, C. G. Hartinger, W. Berger and P. Heffeter, Anticancer activity of metal complexes: involvement of redox processes, Antioxid. Redox Signaling, 2011, 15, 1085-1127
16. N. J. Farrer, L. Salassa and P. J. Sadler, Photoactivated chemotherapy (PACT): the potential of excited-state d-block metals in medicine, Dalton Trans., 2009, 10690-10701
17. K. D. Mjos and C. Orvig, Metallodrugs in Medicinal Inorganic Chemistry, Chem. Rev., 2014, 114, 4540-4563
18. E. S. Antonarakis and A. Emadi, Ruthenium-based chemotherapeutics: are they ready for prime time?, Cancer Chemother. Pharmacol., 2010, 66, 1-9
19. J. M. Rademaker-Lakhai, D. van den Bongard, D. Pluim, J. H. Beijnen and J. H. M. Schellens, A phase I and pharmacological study with imidazolium-trans-DMSO-imidazole-tetrachlororuthenate, a novel ruthenium anticancer agent, Clin. Cancer Res., 2004, 10, 3717-3727
20. K. M. Knopf, B. L. Murphy, S. N. MacMillan, J. M. Baskin, M. P. Barr, E. Boros and J. J. Wilson, In vitro anticancer activity and in vivo biodistribution of rhenium(I) tricarbonyl aqua complexes, J. Am. Chem. Soc., 2017, 139, 14302-14314
21. K. Suntharalingam, S. G. Awuah, P. M. Bruno, T. C. Johnstone, F. Wang, W. Lin, Y. R. Zheng, J. E. Page, M. T. Hemann and S. J. Lippard, Necroptosis-Inducing Rhenium(V) Oxo Complexes, J Am. Chem. Soc., 2015, 137, 2967-2974
22. J. M. Hearn, I. Romero-Canelón, A. F. Munro, Y. Fu, A. M. Pizarro, M. J. Garnett, U. McDermott, N. O. Carragher and P. J. Sadler, Potent organo-osmium compound shifts metabolism in epithelial ovarian cancer cells, Proc. Natl. Acad. Sci. U.S.A, 2015, 112, E3800-E3805
23. K. Suntharalingam, T. C. Johnstone, P. M. Bruno, W. Lin, M. T. Hemann and S. J. Lippard, Bidentate Ligands on Osmium(VI) Nitrido Complexes Control Intracellular Targeting and Cell Death Pathways, J Am. Chem. Soc., 2013, 135, 14060-14063
24. R. T. Mertens, S. Parkin and S. G. Awuah, Cancer cell-selective modulation of mitochondrial respiration and metabolism by potent organogold(III) dithiocarbamates, Chem. Sci., 2020, 11, 10465-10482
25. S. Gukathasan, S. Parkin and S. G. Awuah, Cyclometalated Gold(III) Complexes Bearing DACH Ligands, Inorg. Chem., 2019, 58, 9326-9340
26. J. H. Kim, E. Reeder, S. Parkin and S. G. Awuah, Gold(I/III)-Phosphine Complexes as Potent Antiproliferative Agents, Sci. Rep., 2019, 9, 12335
27. I. Ott On the medicinal chemistry of gold complexes as anticancer drugs, Coord. Chem. Rev., 2009, 253, 1670-1681
28. I. Anvarhusein and P. J. Sadler, A carbon-13 nuclear magnetic resonance study of thiol-exchange reactions of gold(I) thiomalate (“Myocrisin”) including applications to cysteine derivatives, J Chem. Soc., Dalton Trans., 1982, 135-141
29. Zou, T. et al. Deubiquitinases as Anticancer Targets of Gold Complexes. Israel Journal of Chemistry 56, 825-833, doi: 10.1002/ijch.201600044 (2016).
30. S. Preiß, C. Förster, S. Otto, M. Bauer, P. Müller, D. Hinderberger, H. Hashemi Haeri, L. Carella and K. Heinze, Structure and reactivity of a mononuclear gold(II) complex, Nat. Chem., 2017, 9, 1249
31. C. Y. Wu, T. Horibe, C. B. Jacobsen and F. D. Toste, Stable gold(III) catalysts by oxidative addition of a carbon-carbon bond, Nature, 2015, 517, 449-454
32. B. Bertrand, S. Spreckelmeyer, E. Bodio, F. Cocco, M. Picquet, P. Richard, P. Le Gendre, C. Orvig, M. A. Cinellu and A. Casini, Exploring the potential of gold(III) cyclometallated compounds as cytotoxic agents: variations on the C\hat{ }N theme, Dalton Trans., 2015, 44, 11911-11918
33. S. Carboni, A. Zucca, S. Stoccoro, L. Maiore, M. Arca, F. Ortu, C. Artner, B. K. Keppler, S. M. Meier-Menches, A. Casini and M. A. Cinellu, New Variations on the Theme of Gold(III) C(wedge)N(wedge)N Cyclometalated Complexes as Anticancer Agents: Synthesis and Biological Characterization, Inorg. Chem., 2018, 57, 14852-14865
34. G. Marcon, S. Carotti, M. Coronnello, L. Messori, E. Mini, P. Orioli, T. Mazzei, M. A. Cinellu and G. Minghetti, Gold(III) complexes with bipyridyl ligands: solution chemistry, cytotoxicity, and DNA binding properties, J Med. Chem., 2002, 45, 1672-1677
35. L. Massai, D. Cirri, E. Michelucci, G. Bartoli, A. Guerri, M. A. Cinellu, F. Cocco, C. Gabbiani and L. Messori, Organogold(III) compounds as experimental anticancer agents: chemical and biological profiles, BioMetals, 2016, 29, 863-872
36. L. Messori, G. Marcon, M. A. Cinellu, M. Coronnello, E. Mini, C. Gabbiani and P. Orioli, Solution chemistry and cytotoxic properties of novel organogold(III) compounds, Bioorg. Med. Chem., 2004, 12, 6039-6043
37. R. T. Mertens, S. R. Parkin and S. G. Awuah, Synthesis and crystal structure of 1,3-bis-(4-hy-droxy-phen-yl)-1H-imidazole-3-ium chloride, Acta Crystallogr., Sect. E: Crystallogr. Commun., 2019, 75, 1311-1315
38. J. H. Kim, R. T. Mertens, A. Agarwal, S. Parkin, G. Berger and S. G. Awuah, Direct intramolecular carbon(sp(2))-nitrogen(sp(2)) reductive elimination from gold(III), Dalton Trans., 2019, 48, 6273-6282
39. R. T. Mertens, J. H. Kim, W. C. Jennings, S. Parkin and S. G. Awuah, Revisiting the reactivity of tetrachloroauric acid with N,N-bidentate ligands: structural and
spectroscopic insights, Dalton Trans., 2019, 48, 2093-2099
40. D. Hu, Y. Liu, Y. T. Lai, K. C. Tong, Y. M. Fung, C. N. Lok and C. M. Che, Anticancer Gold(III) Porphyrins Target Mitochondrial Chaperone Hsp60, Angew. Chem., Int. Ed., 2016, 55, 1387-1391
41. C. M. Che, R. W. Y. Sun, W. Y. Yu, C. B. Ko, N. Zhu and H. Sun, Gold(III) porphyrins as a new class of anticancer drugs: cytotoxicity, DNA binding and induction of apoptosis in human cervix epitheloid cancer cells, Chem. Commun., 2003, 1718-1719
42. A. D. Lammer, M. E. Cook and J. L. Sessler, Synthesis and anti-cancer activities of a water soluble gold(III) porphyrin, J. Porphyrins Phthalocyanines, 2015, 19, 398-403
43. K. C. Tong, D. Hu, P. K. Wan, C. N. Lok and C. M. Che, Anticancer Gold(III) Compounds with Porphyrin or N-heterocyclic Carbene Ligands, Front. Chem., 2020, 8, 587207
44. I. Toubia, C. Nguyen, S. Diring, L. M. A. Ali, L. Larue, R. Aoun, C. Frochot, M. Gary-Bobo, M. Kobeissi and F. Odobel, Synthesis and Anticancer Activity of Gold Porphyrin Linked to Malonate Diamine Platinum Complexes, Inorg. Chem., 2019, 58, 12395-12406
45. M. Serratrice, M. A. Cinellu, L. Maiore, M. Pilo, A. Zucca, C. Gabbiani, A. Guerri, I. Landini, S. Nobili, E. Mini and L. Messori, Synthesis, Structural Characterization, Solution Behavior, and in Vitro Antiproliferative Properties of a Series of Gold Complexes with 2-(2′-Pyridyl)benzimidazole as Ligand: Comparisons of Gold(III) versus Gold(I) and Mononuclear versus Binuclear Derivatives, Inorg. Chem., 2012, 51, 3161-3171
46. J. R. Stenger-Smith and P. K. Mascharak, Gold Drugs with {Au(PPh₃)}+ Moiety: Advantages and Medicinal Applications, ChemMedChem, 2020, 15, 2136-2145
47. T. Srinivasa Reddy, S. H. Privér, V. V. Rao, N. Mirzadeh and S. K. Bhargava, Gold(I) and gold(III) phosphine complexes: synthesis, anticancer activities towards 2D and 3D cancer models, and apoptosis inducing properties, Dalton Trans., 2018, 47, 15312-15323
48. M. A. Cinellu, A. Zucca, S. Stoccoro, G. Minghetti, M. Manassero and M. Sansoni, Synthesis and characterization of gold(III) adducts and cyclometallated derivatives with 2-substituted pyridines. Crystal structure of [Au{NC₅H₄(CMe₂C₆H₄)—2}Cl₂], J. Chem. Soc., Dalton Trans., 1995, 2865-2872
49. S. J. Berners-Price, C. K. Mirabelli, R. K. Johnson, M. R. Mattern, F. L. McCabe, L. F. Faucette, C. M. Sung, S. M. Mong, P. J. Sadler and S. T. Crooke, In vivo antitumor activity and in vitro cytotoxic properties of bis[1,2-bis(diphenylphosphino)ethane]gold(I) chloride, Cancer Res., 1986, 46, 5486-5493
50. O. Rackham, S. J. Nichols, P. J. Leedman, S. J. Berners-Price and A. Filipovska, A gold(I) phosphine complex selectively induces apoptosis in breast cancer cells: implications for anticancer therapeutics targeted to mitochondria, Biochem. Pharmacol., 2007, 74, 992-1002
51. S. J. Berners-Price, G. R. Girard, D. T. Hill, B. M. Sutton, P. S. Jarrett, L. F. Faucette, R. K. Johnson, C. K. Mirabelli and P. J. Sadler, Cytotoxicity and antitumor activity of some tetrahedral bis(diphosphino)gold(I) chelates, J Med. Chem., 1990, 33, 1386-1392
52. R. G. Buckley, A. M. Elsome, S. P. Fricker, G. R. Henderson, B. R. C. Theobald, R. V. Parish, B. P. Howe and L. R. Kelland, Antitumor Properties of Some 2-[(Dimethylamino)methyl]phenylgold(III) Complexes, J. Med. Chem., 1996, 39, 5208-5214
53. H. Q. Liu, T. C. Cheung, S. M. Peng and C. M. Che, Novel luminescent cyclometaiated and terpyridine gold(III) complexes and DNA binding studies, J Chem. Soc., Chem. Commun., 1995, 17, 1787-1788
54. S. Carboni, A. Zucca, S. Stoccoro, L. Maiore, M. Arca, F. Ortu, C. Artner, B. K. Keppler, S. M. Meier-Menches, A. Casini and M. A. Cinellu, New Variations on the Theme of Gold(III) C\hat{ }N\hat{ }N Cyclometalated Complexes as Anticancer Agents: Synthesis and Biological Characterization, Inorg. Chem., 2018, 57, 14852-14865
55. M. Frik, J. Fernández-Gallardo, O. Gonzalo, V. Mangas-Sanjuan, M. González-Alvarez, A. Serrano del Valle, C. Hu, I. González-Alvarez, M. Bermejo, I. Marzo and M. Contel, Cyclometalated Iminophosphorane Gold(III) and Platinum(II) Complexes. A Highly Permeable Cationic Platinum(II) Compound with Promising Anticancer Properties, J. Med. Chem., 2015, 58, 5825-5841
56. S. K. Fung, T. Zou, B. Cao, P. Y. Lee, Y. M. Fung, D. Hu, C. N. Lok and C. M. Che, Cyclometalated Gold(III) Complexes Containing N-Heterocyclic Carbene Ligands Engage Multiple Anti-Cancer Molecular Targets, Angew. Chem., 2017, 56, 3892-3896
57. L. Massai, D. Cirri, E. Michelucci, G. Bartoli, A. Guerri, M. A. Cinellu, F. Cocco, C. Gabbiani and L. Messori, Organogold(III) compounds as experimental anticancer agents: chemical and biological profiles, BioMetals, 2016, 29, 863-872
58. R. Kumar and C. Nevado, Cyclometalated Gold(III) Complexes: Synthesis, Reactivity, and Physicochemical Properties, Angew. Chem., Int. Ed., 2017, 56, 1994-2015
59. S. Gukathasan, S. Parkin and S. G. Awuah, Cyclometalated Gold(III) Complexes Bearing DACH Ligands, Inorg. Chem., 2019, 58, 9326-9340
60. E. K. Dennis, J. H. Kim, S. Parkin, S. G. Awuah and S. Gameau-Tsodikova, Distorted Gold(I)-Phosphine Complexes as Antifungal Agents, J. Med. Chem., 2020, 63, 2455-2469
61. D. Jia, M. Lu, K. H. Jung, J. H. Park, L. Yu, J. N. Onuchic, B. A. Kaipparettu and H. Levine, Elucidating cancer metabolic plasticity by coupling gene regulation with metabolic pathways, Proc. Natl. Acad. Sci. U.S.A, 2019, 116, 3909-3918
62. J. H. Park, S. Vithayathil, S. Kumar, P. L. Sung, L. E. Dobrolecki, V. Putluri, V. B. Bhat, S. K. Bhowmik, V. Gupta, K. Arora, D. Wu, E. Tsouko, Y. Zhang, S. Maity, T. R. Donti, B. H. Graham, D. E. Frigo, C. Coarfa, P. Yotnda, N. Putluri, A. Sreekumar, M. T. Lewis, C. J. Creighton, L. J. C. Wong and B. A. Kaipparettu, Fatty Acid Oxidation-Driven Src Links Mitochondrial Energy Reprogramming and Oncogenic Properties in Triple-Negative Breast Cancer, Cell Rep., 2016, 14, 2154-2165
63. V. S. LeBleu, J. T. O'Connell, K. N. Gonzalez Herrera, H. Wikman, K. Pantel, M. C. Haigis, F. M. de Carvalho, A. Damascena, L. T. Domingos Chinen, R. M. Rocha, J. M. Asara and R. Kalluri, PGC-1α mediates mitochondrial biogenesis and oxidative phosphorylation in cancer cells to promote metastasis, Nat. Cell Biol., 2014, 16, 992-1003
64. P. E. Porporato, V. L. Payen, J. Pérez-Escuredo, C. J. De Saedeleer, P. Danhier, T. Copetti, S. Dhup, M. Tardy, T. Vazeille, C. Bouzin, O. Feron, C. Michiels, B. Gallez and P. Sonveaux, A Mitochondrial Switch Promotes Tumor Metastasis, Cell Rep., 2014, 8, 754-766
65. R. Camarda, A. Y. Zhou, R. A. Kohnz, S. Balakrishnan, C. Mahieu, B. Anderton, H. Eyob, S. Kajimura, A. Tward, G. Krings, D. K. Nomura and A. Goga, Inhibition of fatty acid oxidation as a therapy for MYC-overexpressing triple-negative breast cancer, Nat. Med., 2016, 22, 427-432
66. S. E. Weinberg and N. S. Chandel, Targeting mitochondria metabolism for cancer therapy, Nat. Chem. Biol., 2015, 11, 9-15
67. J. Lee, A. E. Yesilkanal, J. P. Wynne, C. Frankenberger, J. Liu, J. Yan, M. Elbaz, D. C. Rabe, F. D. Rustandy, P. Tiwari, E. A. Grossman, P. C. Hart, C. Kang, S. M. Sanderson, J. Andrade, D. K. Nomura, M. G. Bonini, J. W. Locasale and M. R. Rosner, Effective breast cancer combination therapy targeting BACH1 and mitochondrial metabolism, Nature, 2019, 568, 254-258
68. G. Libby, L. A. Donnelly, P. T. Donnan, D. R. Alessi, A. D. Morris and J. M. M. Evans, New users of metformin are at low risk of incident cancer. A cohort study among people with type 2 diabetes, Diabetes Care, 2009, 32, 1620-1625
69. W. W. Wheaton, S. E. Weinberg, R. B. Hamanaka, S. Soberanes, L. B. Sullivan, E. Anso, A. Glasauer, E. Dufour, G. M. Mutlu, G. R. S. Budigner and N. S. Chandel,
Metformin inhibits mitochondrial complex I of cancer cells to reduce tumorigenesis, eLife, 2014, 3, e02242
70. A. Naguib, G. Mathew, C. R. Reczek, K. Watrud, A. Ambrico, T. Herzka, I. C. Salas, M. F. Lee, N. El-Amine, W. Zheng, M. E. Di Francesco, J. R. Marszalek, D. J. Pappin, N. S. Chandel and L. C. Trotman, Mitochondrial Complex I Inhibitors Expose a Vulnerability for Selective Killing of Pten-Null Cells, Cell Rep., 2018, 23, 58-67
71. J. R. Molina, Y. Sun, M. Protopopova, S. Gera, M. Bandi, C. Bristow, T. McAfoos, P. Morlacchi, J. Ackroyd, A. N. A. Agip, G. Al-Atrash, J. Asara, J. Bardenhagen, C. C. Carrillo, C. Carroll, E. Chang, S. Ciurea, J. B. Cross, B. Czako, A. Deem, N. Daver, J. F. de Groot, J. W. Dong, N. Feng, G. Gao, J. Gay, M. G. Do, J. Greer, V. Giuliani, J. Han, L. Han, V. K. Henry, J. Hirst, S. Huang, Y. Jiang, Z. Kang, T. Khor, S. Konoplev, Y. H. Lin, G. Liu, A. Lodi, T. Lofton, H. Ma, M. Mahendra, P. Matre, R. Mullinax, M. Peoples, A. Petrocchi, J. Rodriguez-Canale, R. Serreli, T. Shi, M. Smith, Y. Tabe, J. Theroff, S. Tiziani, Q. Xu, Q. Zhang, F. Muller, R. A. DePinho, C. Toniatti, G. F. Draetta, T. P. Heffernan, M. Konopleva, P. Jones, M. E. Di Francesco and J. R. Marszalek, An inhibitor of oxidative phosphorylation exploits cancer vulnerability, Nat. Med., 2018, 24, 1036-1046
72. X. Liu, I. L. Romero, L. M. Litchfield, E. Lengyel and J. W. Locasale, Metformin Targets Central Carbon Metabolism and Reveals Mitochondrial Requirements in Human Cancers, CellMetab., 2016, 24, 728-739
73. S. R. Lord, W. C. Cheng, D. Liu, E. Gaude, S. Haider, T. Metcalf, N. Patel, E. J. Teoh, F. Gleeson, K. Bradley, S. Wigfield, C. Zois, D. R. McGowan, M. L. Ah-See, A. M. Thompson, A. Sharma, L. Bidaut, M. Pollak, P. G. Roy, F. Karpe, T. James, R. English, R. F. Adams, L. Campo, L. Ayers, C. Snell, I. Roxanis, C. Frezza, J. D. Fenwick, F. M. Buffa and A. L. Harris, Integrated Pharmacodynamic Analysis Identifies Two Metabolic Adaption Pathways to Metformin in Breast Cancer, Cell Metab., 2018, 28, 679-688
74. Y. Shi, S. K. Lim, Q. Liang, S. V. Iyer, H. Y. Wang, Z. Wang, X. Xie, D. Sun, Y. J. Chen, V. Tabar, P. Gutin, N. Williams, J. K. De Brabander and L. F. Parada, Gboxin is an oxidative phosphorylation inhibitor that targets glioblastoma, Nature, 2019, 567, 341-346
75. K. D. Paull, R. H. Shoemaker, L. Hodes, A. Monks, D. A. Scudiero, L. Rubinstein, J. Plowman and M. R. Boyd, Display and analysis of patterns of differential activity of drugs against human tumor cell lines: development of mean graph and COMPARE algorithm, J. Natl. Cancer Inst., 1989, 81, 1088-1092
76. R. H. Shoemaker The NCI60 human tumour cell line anticancer drug screen, Nat. Rev. Cancer, 2006, 6, 813-823
77. M. Kanehisa and S. Goto, KEGG: Kyoto Encyclopedia of Genes and Genomes, Nucleic Acids Res., 2000, 28, 27-30
78. M. Kanehisa Toward understanding the origin and evolution of cellular organisms, Protein Sci., 2019, 28, 1947-1951
79. M. Kanehisa, Y. Sato, M. Furumichi, K. Morishima and M. Tanabe, New approach for understanding genome variations in KEGG, Nucleic Acids Res., 2018, 47, D590-D595.
80. Bruker, “APEX2” Bruker-AXS. Madison, WI. USA, 2016.
81. Krause, L.; Herbst-Irmer, R.; Sheldrick, G. M.; Stalke, D., Comparison of silver and
molybdenum microfocus X-ray sources for single-crystal structure determination. J Appl Crystallogr 2015, 48 (Pt 1), 3,10.
82. Sheldrick, G. M., SADABS, Program for bruker area detector absorption correction. University of Gottingen, Gottingen, 1997.
83. Sheldrick, G. M., Crystal structure refinement with SHELXL. Acta Crystallogr C Struct Chem 2015, 71 (Pt 1), 3-8.
84. Sheldrick, G. M., SHELXT-integrated space-group and crystal-structure determination. Acta Crystallogr A Found Adv 2015, 71 (Pt 1), 3-8.
85. Parkin, S., Expansion of scalar validation criteria to three dimensions: the R tensor. Erratum. Acta Crystallogr A 2000, 56 (Pt 3), 317.
86. Spek, A. L., Structure validation in chemical crystallography. Acta Crystallogr D Biol Crystallogr 2009, 65 (Pt 2), 148-55.
87. Wu, Y. et al. Dub3 inhibition suppresses breast cancer invasion and metastasis by promoting Snail1 degradation. Nature Communications 8, 14228, doi:10.1038/ncomms14228 (2017).
88. Bianchini, G., Balko, J. M., Mayer, I. A., Sanders, M. E. & Gianni, L. Triple-negative breast cancer: challenges and opportunities of a heterogeneous disease. Nat Rev Clin Oncol 13, 674-690, doi:10.1038/nrclinonc.2016.66 (2016).
89. Lehmann, B. D. et al. Identification of human triple-negative breast cancer subtypes and preclinical models for selection of targeted therapies. J Clin Invest 121, 2750-2767, doi:10.1172/JC145014 (2011).
90. Isakoff, S. J. Triple-negative breast cancer: role of specific chemotherapy agents. Cancer J16, 53-61, doi:10.1097/PPO.0b013e3181d24ff7 (2010).
91. Gucalp, A. & Traina, T. A. Triple-negative breast cancer: adjuvant therapeutic options. Chemother Res Pract 2011, 696208, doi: 10.1155/2011/696208 (2011).
92. Schmid, P. et al. Atezolizumab and Nab-Paclitaxel in Advanced Triple-Negative Breast Cancer. N Engl J Med 379, 2108-2121, doi:10.1056/NEJMoa1809615 (2018).
93. Holohan, C., Van Schaeybroeck, S., Longley, D. B. & Johnston, P. G. Cancer drug resistance: an evolving paradigm. Nat Rev Cancer 13, 714-726, doi:10.1038/nrc3599 (2013).
94. Hickey, J. L. et al. Mitochondria-Targeted Chemotherapeutics: The Rational Design of Gold(I)N-Heterocyclic Carbene Complexes That Are Selectively Toxic to Cancer Cells and Target Protein Selenols in Preference to Thiols. Journal of the American Chemical Society 130, 12570-12571, doi:10.1021/ja804027j (2008).
95. (US), C. g. I B. M. N. L. o. M. (2011 September-2016 September Identifier NCT01419691).
96. (US), C. g. I B. M. N. L. o. M. Auranofin in Decreasing Pain in Patients With Paclitaxel-Induced Pain Syndrome. (2014 February-2016 November Identifier NCT02063698).
97. Graham, G. G., Champion, G. D. & Ziegler, J. B. The Cellular Metabolism and Effects of Gold Complexes. Metal-Based Drugs 1, 395-404, doi:10.1155/MBD.1994.395 (1994).
98. Zhang, Y. et al. Gold binding sites in red blood cells. Inorganica ChimicaActa 229, 271-280, doi:doi.org/10.1016/0020-1693(94)04254-S (1995).
99. Berners-Price, S. J. S., P. J. in Structure and Bonding 70 1-76 (Springer-Verlag, 1988).
100. Huang, H. et al. Two clinical drugs deubiquitinase inhibitor auranofin and aldehyde dehydrogenase inhibitor disulfiram trigger synergistic anti-tumor effects in vitro and in vivo. Oncotarget 7, 2796-2808, doi:10.18632/oncotarget.6425 (2016).
101. Zhang, J. J., Ng, K. M., Lok, C. N., Sun, R. W. Y. & Che, C. M. Deubiquitinases as potential anti-cancer targets for gold(iii) complexes. Chemical Communications 49, 5153-5155, doi:10.1039/C3CC41766B (2013).
102. Milacic, V. & Dou, Q. P. The tumor proteasome as a novel target for gold(III) complexes: implications for breast cancer therapy. Coordination chemistry reviews 253, 1649-1660, doi:10.1016/j.ccr.2009.01.032 (2009).
103. Schuh, E. et al. Gold(I) Carbene Complexes Causing Thioredoxin 1 and Thioredoxin 2 Oxidation as Potential Anticancer Agents. Journal of Medicinal Chemistry 55, 5518-5528, doi:10.1021/jm300428v (2012).
104. Bindoli, A. et al. Thioredoxin reductase: A target for gold compounds acting as potential anticancer drugs. Vol. 253 (2009).
105. Deponte, M. et al. Mechanistic studies on a novel, highly potent gold-phosphole inhibitor of human glutathione reductase. J Biol Chem 280, 20628-20637, doi:10.1074/jbc.M412519200 (2005).
106. Urig, S. et al. Undressing of phosphine gold(I) complexes as irreversible inhibitors of human disulfide reductases. Angew Chem Int Ed Engl 45, 1881-1886, doi:10.1002/anie.200502756 (2006).
107. Rohozková, D. & Steven, F. S. Gold-containing drugs and the control of proteolytic enzymes. British Journal of Pharmacology 79, 181-189, doi:10.1111/j.1476-5381.1983.tb10511.x (1983).
108. Tian, S., Siu, F. M., Kui, S. C. F., Lok, C. N. & Che, C. M. Anticancer gold(i)-phosphine complexes as potent autophagy-inducing agents. Chemical Communications 47, 9318-9320, doi:10.1039/C1CC11820J (2011).
109. Roisman, F. R., Walz, D. T., Finkelstein, A. E. Superoxide radical production by human leukocytes exposed to immune complexes: inhibitory action of gold compounds. Inflammation 7, 355-362 (1983).

It will be understood that various details of the presently disclosed subject matter can be changed without departing from the scope of the subject matter disclosed herein. Furthermore, the foregoing description is for the purpose of illustration only, and not for the purpose of limitation.

1. A compound having the following formula or a pharmaceutically acceptable salt thereof: 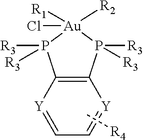
wherein R₁ and R₂, taken together with the gold (Au) to which they are bound, form a polycyclic moiety selected from the group consisting of: 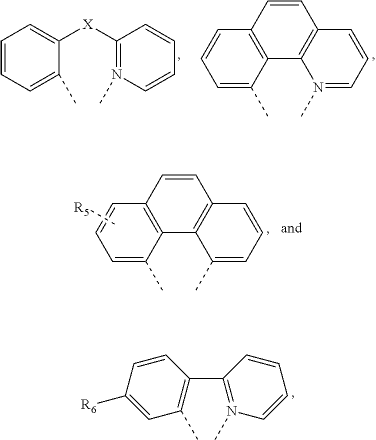
and
X is CH₂, O, NH, or C═O;
Y is C or N;
each R₃ is independently selected from the group consisting of H, alkyl, and aryl;
R₄ is H, alkyl, aryl, or taken together with two of the atoms of the ring to which it is bound, forms a 6-membered ring;
R₅ is H, alkyl, or aryl; and
R₆ is H, CH₃, COH, a targeting ligand, or an affinity tag.

2. The compound according to claim 1, wherein R₆ is a targeting ligand.

3. The compound according to claim 1, wherein R₆ is an affinity tag.

4. The compound of claim 1, having the structure: 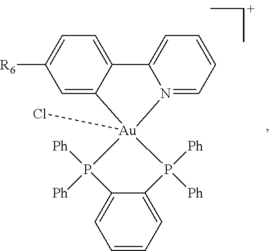
wherein R₆ is selected from the group consisting of CH₃, COH, and H.

5. The compound of claim 1, having the structure: 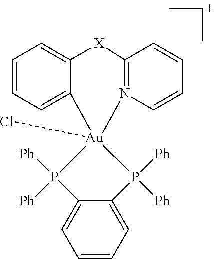
wherein X is selected from the group consisting of CH₂, O, NH, and C═O.

6. The compound of claim 1, having the structure:

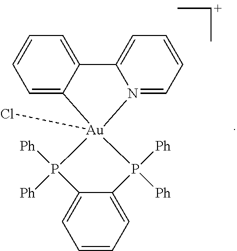

7. The compound of claim 1, having the structure:

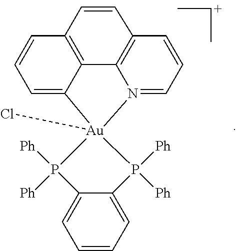

8. The compound of claim 1, having the structure:

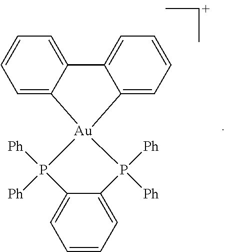

9. The compound of claim 1, having the structure:

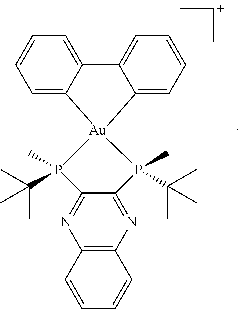

10. A pharmaceutical composition comprising the compound of claim 1 and a pharmaceutically-acceptable carrier.

11. A method of conferring anti-cancer activity to a cancer cell, comprising: contacting a cancer cell with an effective amount of the compound of claim 1.

12. The method of claim 11, wherein the conferring anti-cancer activity results in one or more of inhibiting proliferation of the cancer cell, inhibiting metastasis, and killing the cancer cell.

13. The method of claim 11, wherein the cell is a cultured cell.

14. The method of claim 11, wherein the cell is in a subject.

15. The method of claim 14, wherein the subject is a mammal.

16. A method of modulating mitochondrial function in a cell, comprising: contacting a cell with an effective amount of the compound of claim 1.

17. The method of claim 16, wherein the cell is a cancer cell.

18. The method of claim 16, wherein the cell is a cultured cell.

19. The method of claim 16, wherein the cell is in a subject.

20. The method of claim 43, wherein the subject is a mammal.

21. (canceled)

22. (canceled)
